(19) United States
(12) Patent Application Publication
(10) Pub. No.: US 2020/0247808 A1
(43) Pub. Date: Aug. 6, 2020

(54) Heterocyclic Tec-Family Kinase Inhibitors

(71) Applicant: GB005, Inc., San Diego, CA (US)

(72) Inventors: Alain Laurent, Montreal (CN); Yannick Rose, Montreal (CA); Stephen J. Morris, Montreal (CA)

(21) Appl. No.: 15/772,916

(22) PCT Filed: Sep. 9, 2016
(86) PCT No.: PCT/CA2016/051068

§ 371 Date: May 2, 2018

(30) Foreign Application Priority Data
Sep. 11, 2015 (CA) 2.904.270

(51) Int. Cl. C07D 487/04; A61P 31/18

(52) U.S. Cl. CPC C07D 487/04; A61K 45/06; A61P 31/18

(57) ABSTRACT

Compounds having the Formula (I), and enantiomers, diastereomers, pharmaceutically acceptable salts, solvates and solvates of salts thereof, (Formula I)) are useful as kinase inhibitors or modulators, including BTK modulation or inhibition, wherein X¹, X², m, m′, E and R¹ are as defined herein.

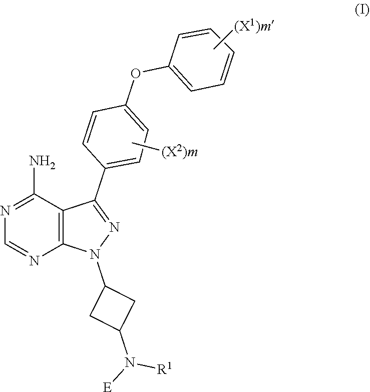

### FIELD OF INVENTION

The present invention relates to a novel family of protein kinase inhibitors, to pharmacological compositions that contain them and uses of the inhibitors to treat or prevent diseases, disorders and conditions associated with kinase function.

### BACKGROUND OF THE INVENTION

Protein kinases are a large group of intracellular and transmembrane signalling proteins in eukaryotic cells (Manning G. et al, (2002) Science, 298: 1912-1934). These enzymes are responsible for transfer of the terminal (gamma) phosphate from ATP to specific amino acid residues of target proteins. Phosphorylation of specific amino acid residues in target proteins can modulate their activity leading to profound changes in cellular signalling and metabolism. Protein kinases can be found in the cell membrane, cytosol and organelles such as the nucleus and are responsible for mediating multiple cellular functions including metabolism, cellular growth and differentiation, cellular signalling, modulation of immune responses, and cell death. Serine kinases specifically phosphorylate serine or threonine residues in target proteins. Similarly, tyrosine kinases, including tyrosine receptor kinases, phosphorylate tyrosine residues in target proteins. Tyrosine kinase families include: TEC, SRC, ABL, JAK, CSK, FAK, SYK, FER, ACK and the receptor tyrosine kinase subfamilies including ERBB, FGFR, VEGFR, RET and EPH. Subclass I of the receptor tyrosine kinase superfamily consists of the ERBB receptors and comprises four members: ErbB1 (also called epidermal growth factor receptor (EGFR)), ErbB2, ErbB3 and ErbB4.

Kinases exert control on key biological processes related to health and disease. Furthermore, aberrant activation or excessive expression of various protein kinases are implicated in the mechanism of multiple diseases and disorders characterized by benign and malignant proliferation, as well as diseases resulting from inappropriate activation of the immune system (Kyttaris V C, Drug Des Devel Ther, 2012, 6:245-50 and Fabbro D. et al. Methods Mol Biol, 2012, 795:1-34). Thus, inhibitors of select kinases or kinase families may be useful in the treatment of cancer, vascular disease, autoimmune diseases, and inflammatory conditions including, but not limited to: solid tumors, hematological malignancies, thrombus, arthritis, graft versus host disease, lupus erythematosus, psoriasis, colitis, illeitis, multiple sclerosis, uveitis, coronary artery vasculopathy, systemic sclerosis, atherosclerosis, asthma, transplant rejection, allergy, ischemia, dermatomyositis, pemphigus, and the like.

Tec kinases are a family of non-receptor tyrosine kinases predominantly, but not exclusively, expressed in cells of hematopoietic origin (Bradshaw J M. Cell Signal. 2010,22:1175-84). The Tec family includes TEC, Bruton's tyrosine kinase (BTK), inducible T-cell kinase (ITK), resting lymphocyte kinase (RLK/TXK for Tyrosine Protein Kinase), and bone marrow-expressed kinase (BMX/ETK).

BTK is important in B-cell receptor signaling and regulation of B-cell development and activation (W. N. Khan et al. Immunity, 1995,3:283-299 and Satterthwaite A B et al. Immunol. Rev. 2000,175: 120-127). Mutation of the gene encoding BTK in humans leads to X-linked agammaglobulinemia which is characterized by reduced immune function, including impaired maturation of B-cells, decreased levels of immunoglobulin and peripheral B cells, diminished T-cell independent immune response (Rosen F S et al., N Engl. J. Med., 1995, 333:431-440; and Lindvall J M et al. Immunol. Rev. 2005,203:200-215). BTK is activated by Src-family kinases and phosphorylates PLC gamma leading to effects on B-cell function and survival. Additionally, BTK is important for cellular function of mast cells, macrophage and neutrophils suggesting that BTK inhibition would be effective in treatment of diseases mediated by these and related cells including inflammation, bone disorders, and allergic disease (Kawakami Y. et al., J Leukoc Biol. 1999;65(3):286-90). BTK inhibition is also important in survival of lymphoma cells (Herman S E M. Blood, 2011, 117:6287-6289) suggesting that inhibition of BTK may be useful in the treatment of lymphomas and other cancers (Uckun F M, Int Rev Immunol. 2008;27(1-2):43-69). As such, inhibitors of BTK and related kinases are of great interest as anti-inflammatory as well as anti-cancer agents. BTK is also important for platelet function and thrombus formation suggesting that BTK-selective inhibitors may prove to be useful antithrombotic agents (Liu J. Blood, 2006,108:2596-603). Furthermore, BTK is required for inflammasome activation and inhibition of BTK may be useful in treatment of inflammasome-related disorders including; stroke, gout, type 2 diabetes, obesity-induced insulin resistance, atherosclerosis and Muckle-Wells syndrome. In addition BTK is expressed in HIV infected T-cells and treatment with BTK inhibitors sensitizes infected cells to apoptotic death and results in decreased virus production (Guendel I et al. J Neurovirol. 2015;21:257-75). Accordingly, BTK inhibitors may be useful in the treatment of HIV-AIDS and other viral infections.

BMX, another Tec family member which has roles in inflammation, cardiovascular disease, and cancer (Cenni B. et al. Int Rev Immuno1.2012, 31: 166-173) is also important for self-renewal and tumorigenic potential of glioblastoma stem cells (Guryanova O A et al. Cancer Cell Cancer Cell 2011,19:498-511). As such, BMX inhibitors may be useful in the treatment of various diseases including cancer, cardiovascular disease and inflammation.

ITK is a key signalling molecule downstream of the T-cell receptor and is expressed in T-cells, mast cells and NK cells (Felices M et al, J. Immunol. 2008;180:3007-3018, Schaeffer E M et al, Science 1999;284:638-641). Inhibition of ITK has been shown to affect cytokine secretion and polarization of T-cell subtypes. As such ITK inhibitors may be useful in the treatment of allergy, psoriasis, dermatitis, multiple sclerosis and other diseases (Kaur M et al. Eur. J. Pharm. Sci. 2013;47:574-588, Kannan A K et al. J. Neurosci. 2015;35:221-233).

Ibrutinib (PCI-32765, Imbruvica) is a highly potent BTK inhibitor approved by the FDA for the treatment of Waldenström's Macroglobulinemia, Chronic Lymphocytic Leukemia, Mantle Cell Lymphoma with potential in other indications. Ibrutinib targets BTK and other members of the Tec family as well as select other kinases (Honigberg L A et al. Proc. Natl. Acad. Sci. 2010;107:13075-13080).

Adverse effects of Ibrutinib, consistent with off-target effects, include diarrhea (Byrd J C et al. N Engl J Med. 2014;371:213-23, O'Brien S et al. Lancet Oncol. 2014;15:48-58, Wang ML et al. Blood. 2015; 9--03-635326), atrial fibrillation (Treon S P et al. N Engl J Med. 2015;372:1430-40, Kim E S et al. Drugs. 2015;75:769-76), hypertension (George B. et al. 2014; Blood: 124 (21)) as well as panniculitis (Fabbro S K et al. JAMA Oncology 2015: doi: 10.1001). As such, the identification of BTK inhibitors with increased safety and tolerability is highly desired.

Additionally, the therapeutic dose of Ibrutinib is elevated, with recommended daily doses as high as 560 mg (four 140 mg capsules) taken orally once daily. Data indicates that idiosyncratic drug toxicities are more likely to occur with high dose (>100mg) drugs (Lammert C. Hepatology 2008;47:2003-2009). Accordingly, BTK inhibitors with improved human or animal pharmacokinetics, resulting in lower total dose, are highly desired.

### SUMMARY OF THE INVENTION

The present invention relates to a novel family of covalent kinases inhibitors. Compounds of this class have been found to have inhibitory activity against members of the Tec kinase family, particularly BTK and to be more selective than the reference compound defined below. In particular, compounds of the instant invention can have decreased affinity for EGFR, ErbB2 and other kinases. Also, compounds of the instant invention can have improved stability in human liver microsomes and improved pharmacokinetics in rodents suggesting improved bioavailability in human. Additionally, compounds of the instant invention can exhibit decreased formation of glutathione adducts revealing a decreased propensity for non-specific reactions with thiols which can lead to immune reactions.

The present invention is directed to a compound of Formula I:

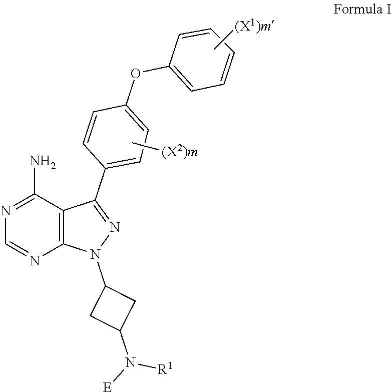

or pharmaceutically acceptable salt, solvate, solvate of salt, stereoisomer, tautomer, isotope, prodrug, complex or biologically active metabolite thereof, wherein
X¹ and X² are independently selected from hydrogen or halogen;
m is an integer from 0 to 4;
m′ is an integer from 0 to 5;
R¹ is selected from hydrogen or a substituted or unsubstituted alkyl;
E is:

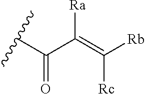

wherein Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb optionally taken together with the carbon atoms to which they are attached form a 3- to 8-membered substituted or unsubstituted cycloalkyl ring, or form a 3- to 8-membered substituted or unsubstituted heterocyclyl ring; or
Rb and Rc optionally can be fused with their intervening atom to form a 3- to 8-membered substituted or unsubstituted cycloalkyl ring, or a 3- to 8-membered substituted or unsubstituted heterocyclyl ring; or
Ra and Rb optionally form a triple bond.

Another embodiment of the present invention includes compounds of Formula II:

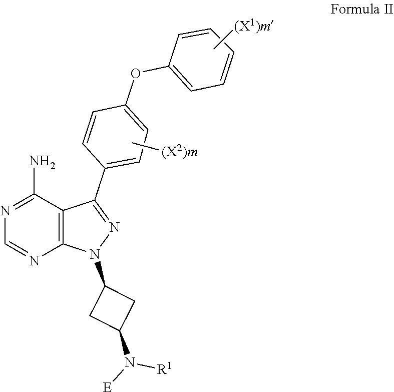

or pharmaceutically acceptable salt, solvate, solvate of salt, stereoisomer, tautomer, isotope, prodrug, complex or biologically active metabolite thereof, wherein
X¹ and X² are independently selected from hydrogen or halogen;
m is an integer from 0 to 4;
m′ is an integer from 0 to 5;
R¹ is selected from hydrogen or a substituted or unsubstituted alkyl;
E is:

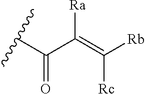

wherein Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb optionally taken together with the carbon atoms to which they are attached form a 3- to 8-membered substituted or unsubstituted cycloalkyl ring, or form a 3- to 8-membered substituted or unsubstituted heterocyclyl ring; or
Rb and Rc optionally can be fused with their intervening atom to form a 3- to 8-membered substituted or unsubstituted cycloalkyl ring, or a 3- to 8-membered substituted or unsubstituted heterocyclyl ring; or
Ra and Rb optionally form a triple bond.

An embodiment includes compounds of Formula I or Formula II, wherein X¹ and X² are independently selected from hydrogen or fluorine.

An embodiment includes compounds of Formula I or Formula II, wherein X¹ and X² are both hydrogen.

An embodiment includes compounds of Formula I or Formula II, wherein X¹ is fluorine and X² is hydrogen.

An embodiment includes compounds of Formula I or Formula II, wherein X¹ is hydrogen and X² is fluorine.

An embodiment includes compounds of Formula I or Formula II, wherein m is selected from 0, 1 or 2.

An embodiment includes compounds of Formula I or Formula II, wherein m′ is selected from 0, 1 or 2.

An embodiment includes compounds of Formula I or Formula II, wherein R¹ is hydrogen.

An embodiment includes compounds of Formula I or Formula II, R¹ is selected from hydrogen or a substituted or unsubstituted C₁₋₆ alkyl, for example methyl.

An embodiment includes compounds of Formula I or Formula II, wherein R¹ is methyl.

An embodiment includes compounds of Formula I or Formula II, wherein E is:

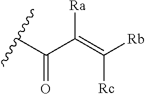

wherein Ra, Rb and Rc are independently selected from hydrogen or substituted or unsubstituted C₁₋₆ alkyl; or
Ra and Rb optionally form a triple bond and Rc is selected from hydrogen or substituted or unsubstituted C₁₋₆ alkyl.

An embodiment includes compounds of Formula I or Formula II, wherein E is selected from the group consisting of:

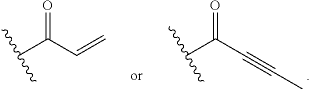

In an alternate embodiment the invention includes compounds of Formula I or Formula II, wherein E is

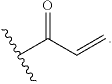 [SMILES: *C(=O)C=C]

In an alternate embodiment the invention includes compounds of Formula I or Formula II, wherein E is

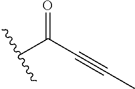 [SMILES: *C(=O)C#CC]

In an embodiment the invention includes compounds of Formula I or Formula II, wherein the compounds are compounds 1 to 7 of Table 1. The compounds in Table 1 are drawn as cis isomers. However, trans isomers and mixture of cis and trans isomers are also contemplated by the present invention.

Another aspect of the present invention provides a pharmaceutical composition comprising a compound of Formula I or Formula II or a pharmaceutically acceptable salt, solvate, solvate of salt, stereoisomer, tautomer, isotope, prodrug, complex or biologically active metabolite thereof and at least one pharmaceutically acceptable carrier, diluent or excipient.

In another aspect, the present invention relates to a compound of the invention as defined herein, or a pharmaceutically acceptable salt or solvate thereof, or a pharmaceutical composition as defined herein, for use in therapy.

In yet another aspect, the present invention relates to a compound of the invention as defined herein, or a pharmaceutically acceptable salt or solvate thereof, or a pharmaceutical composition as defined herein, for use in the treatment of a subject suffering from a protein kinase mediated disease or condition.

In a further aspect of the present invention provides a use of the compound of Formula I or Formula II as an inhibitor of protein kinase, more particularly, as an inhibitor of BTK.

An embodiment of the present invention includes compounds of Formula I or Formula II having improved microsomal stability, increased bioavailability, higher plasma exposure or combinations thereof.

In an alternate embodiment of the present invention includes compounds of Formula I or Formula II that have improved selectivity relative to EGFR and ERB kinases.

In an alternate embodiment of the present invention includes compounds of Formula I that have improved selectivity for BTK relative to EGFR when compared with ibrutinib.

In another embodiment of the present invention, includes compounds of Formula I or Formula II that reduce formation of non-specific thiol adducts relative to the reference compound.

In another aspect, the present invention relates to the use of a compound of the invention as defined herein, or a pharmaceutically acceptable salt or solvate thereof, in the manufacture of a medicament for use in the treatment of subjects suffering from a protein kinase mediated diseases or conditions. Preferably, the present invention relates to the use of a compound of the invention as defined herein, or a pharmaceutically acceptable salt or solvate thereof, in the manufacture of a medicament for use in the treatment of subjects suffering from disease, disorder or condition associated with Tec family members, and BTK kinase activity.

In another aspect, the present invention relates to a method of treating a disease or condition associated with protein kinase activity, said method comprising administering to a subject a therapeutically effective amount of a compound of the invention as defined herein, or a pharmaceutically acceptable salt or solvate thereof, or a pharmaceutical composition as defined herein. Preferably, the present invention further includes a method of treating a disease or condition associated with Tec family members activity, particularly BTK kinase activity, said method comprising administering to a subject a therapeutically effective amount of a compound of the invention as defined herein, or a pharmaceutically acceptable salt or solvate thereof.

In an embodiment of the present invention a compound of Formula I or Formula II, or a pharmaceutically acceptable salt, solvate, solvate of salt, stereoisomer, tautomer, isotope, prodrug, complex or biologically active metabolite thereof, is for use in the treatment or prevention of cancer, autoimmune diseases, allergic diseases, inflammatory diseases, neurological disorders, or viral infection in combination therapy.

In an embodiment of the present invention a compound of Formula I or Formula II, or a pharmaceutically acceptable salt, solvate, solvate of salt, stereoisomer, tautomer, isotope, prodrug, complex or biologically active metabolite thereof, is for use in therapy, further comprising at least one additional active pharmaceutical ingredient for the treatment or prevention of cancer, autoimmune diseases, allergic diseases, inflammatory diseases, neurological disorders or viral infection in combination therapy. The additional active pharmaceutical ingredient is selected from the group consisting of : steroids, leukotriene antagonists, anti-histamines, anti-cancer, anti-viral, anti-biotic agents, protein kinase inhibitors, immune modulators, checkpoint inhibitors and a combination thereof, and wherein additional active pharmaceutical ingredient is administered together with the compounds of Formula I or Formula II, or a pharmaceutically acceptable salt, solvate, solvate of salt, stereoisomer, tautomer, isotope, prodrug, complex or biologically active metabolite thereof, as a single dosage form, or separately as part of a multiple dosage form.

In another aspect, the present invention relates to the use of a compound of the invention as defined herein, or a pharmaceutically acceptable salt or solvate thereof, or a pharmaceutical composition as defined herein, in therapy or prevention of a disease, disorder or condition is associated with Tec family members, and BTK kinase activity.

Compounds of the present invention, in any aspect or embodiment may be used in the treatment or prevention of cancer or autoimmune diseases selected from: rheumatoid arthritis, juvenile rheumatoid arthritis, osteoarthritis, ankylosing spondylitis, psoriatic arthritis, psoriasis vulgaris, pemphigus vulgaris, bullous pemphigoid, Sjogren's syndrome, systemic lupus erythromatosus, discoid SLE, lupus nephritis, antiphospholipidosis, whipple, dermatomyositis, polymyositis, autoimmune thrombocytopenia, idiopathic thrombocytopenia purpura, thrombotic thrombocytopenia purpura, autoimmune (cold) agglutinin disease, autoimmune hemolytic anaemia, cryoglobulinemia, autoimmune vasculitis, ANCA-associated vasculitis, scleroderma, systemic sclerosis, multiple sclerosis, chronic focal encephalitis, Guillian-Barre syndrome, chronic fatigue syndrome, mononucleosis, neuromyelitis optica, autoimmune uveitis, Grave' s disease, thyroid associated opthalmopathy, granulomatosis with microscopic polyangitis, Wegeners granulomatosis, idiopathic pulmonary fibrosis, sarcoidosis, idiopathic membranous nephropathy, IgA nephropathy, glomerulos clerosis , pancreatitis , type I diabetes or type II diabetes, allergic diseases, inflammatory diseases, neurological disorders or viral infection in combination therapy.

Another aspect of the present invention provides a compound, or a pharmaceutically acceptable salt or solvate thereof, or a pharmaceutical composition as defined herein, for use in the treatment of a proliferative disorder, such as cancer.

A further aspect of the present invention provides the use of a compound of Formula I or Formula II, or a pharmaceutically acceptable salt or solvate thereof, in the manufacture of a medicament for the treatment of an autoimmune disease, such as arthritis.

A further aspect of the present invention provides the use of a compound of Formula I or Formula II, or a pharmaceutically acceptable salt or solvate thereof, in the manufacture of a medicament for the treatment of inflammatory diseases, such as lupus.

A further aspect of the present invention provides the use of a compound of Formula I or Formula II, or a pharmaceutically acceptable salt or solvate thereof, in the manufacture of a medicament for the treatment of allergic diseases.

In another aspect, the present invention provides a method of treating a proliferative disorder, said method comprising administering to a subject a therapeutically effective amount of a compound, or a pharmaceutically acceptable salt or solvate thereof, or a pharmaceutical composition as defined herein. In a particular embodiment, the proliferative disorder is a cancer. In an alternate embodiment the method comprises using one or more anticancer agents, anti-inflammatory agents, immunomodulatory agents or combinations thereof in combination with the compounds of the present invention.

Another aspect of the present invention provides a method of modulating kinase function, the method comprising contacting a cell with a compound of the present invention in an amount sufficient to modulate the enzymatic activity of BTK, thereby modulating the kinase function.

Another aspect of the present invention provides a method of inhibiting cell proliferation or survival in vitro or in vivo, said method comprising contacting a cell with an effective amount of a compound as defined herein, or a pharmaceutically acceptable salt or solvate thereof.

In an additional embodiment of the present invention a method of reducing the enzymatic activity of BTK is provided, the method comprising contacting the enzyme with an effective amount of a compound of Formula I or Formula II.

In one embodiment the present invention provides a method of producing a protein kinase inhibitory effect in a cell or tissue, said method comprising contacting the cell or tissue with an effective amount of a compound, or a pharmaceutically acceptable salt or solvate thereof.

In other embodiment, the present invention provides a method of producing a protein kinase inhibitory effect in vivo, said method comprising administering to a subject an effective amount of a compound, or a pharmaceutically acceptable salt or solvate thereof.

Another aspect of the present invention provides a method of modulating the target kinase function, the method comprising:
a) contacting a cell with a compound of the present invention in an amount sufficient to modulate the target kinase function, thereby;
b) modulating the target kinase activity and signaling.

In an embodiment of the present invention, the compounds provided herein are useful for oral, topical, parenteral or intravenous administration.

The present invention further provides a method of synthesizing a compound, or a pharmaceutically acceptable salt or solvate thereof, as defined herein.

Another aspect of the present invention provides a probe, the probe comprising a compound of Formula I or Formula II, labeled with a detectable label or an affinity tag. In other words, the probe comprises a residue of a compound of Formula I or Formula II, covalently conjugated to a detectable label. Such detectable labels include, but are not limited to, a fluorescent moiety, a chemiluminescent moiety, a paramagnetic contrast agent, a metal chelate, a radioactive isotope-containing moiety and biotin.

All publications, patent applications, patents and other references mentioned herein are incorporated by references in their entirety.

Other features, objects, and advantages of the invention(s) disclosed herein will be apparent from the description and drawings, and from the claims.

### BRIEF DESCRIPTION OF THE DRAWINGS

FIG. 1 shows the structure of the reference compound, Compound 13 from Example 1b in patent application WO 2008/039218 A2 also known as 1-((R)-3-(4-amino-3-(4-phenoxyphenyl)-1H-pyrazolo[3,4-d]pyrimidin-1-yl)piperidin-1-yl)prop-2-en-1-one.

FIG. 2 shows that the reference compound more potently inhibits activation of EGFR receptors by EGF than either Compounds 1 or 2. These data suggest that compounds of the instant invention have reduced EGFR-related adverse events compared with the reference compound in vitro.

FIG. 3 shows that Compounds 1 and 2 inhibit immune complex-mediated vasculitis. These data suggest that compounds of the instant invention can be useful in the treatment of diseases and conditions involving deposition of immune complexes and activation of Fc receptors. Such diseases include rheumatoid arthritis and systemic lupus erythematosus.

FIGS. 4 and 5 show that Compounds 1 and 2 are effective in the mouse collagen-induced arthritis model. These data suggest that compounds of the instant invention can be useful in the treatment of rheumatoid arthritis.

FIGS. 6 and 7 show that Compounds 1 and 2 reduce tumor growth in a murine model of lymphoma. These data suggest that compounds of the instant invention can be useful in the treatment of cancer including lymphoma.

### DETAILED DESCRIPTION OF THE INVENTION

The present invention is directed to a compound of Formula I:

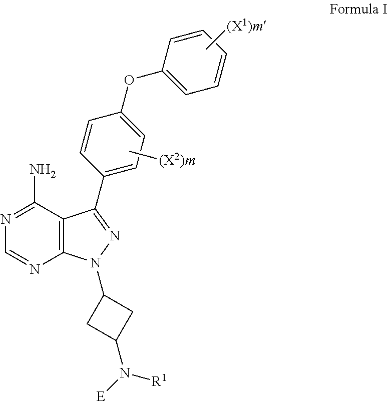

or pharmaceutically acceptable salt, solvate, solvate of salt, stereoisomer, tautomer, isotope, prodrug, complex or biologically active metabolite thereof, wherein
X¹ and X² are independently selected from hydrogen or halogen;
m is an integer from 0 to 4;
m′ is an integer from 0 to 5;
R¹ is selected from hydrogen or a substituted or unsubstituted alkyl;
E is:

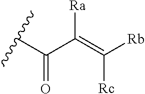

wherein Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb optionally taken together with the carbon atoms to which they are attached form a 3- to 8-membered substituted or unsubstituted cycloalkyl ring, or form a 3- to 8-membered substituted or unsubstituted heterocyclyl ring; or
Rb and Rc optionally can be fused with their intervening atom to form a 3- to 8-membered substituted or unsubstituted cycloalkyl ring, or a 3- to 8- membered substituted or unsubstituted heterocyclyl ring; or
Ra and Rb optionally form a triple bond.

Another embodiment of the present invention is directed to a compound of Formula II:

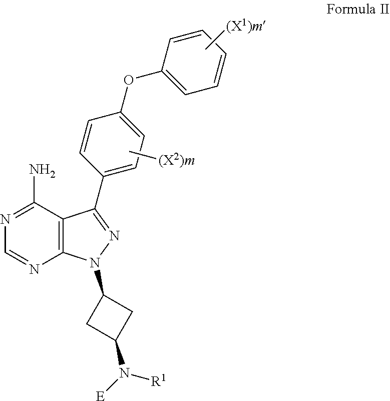

or pharmaceutically acceptable salt, solvate, solvate of salt, stereoisomer, tautomer, isotope, prodrug, complex or biologically active metabolite thereof, wherein
X¹ and X² are independently selected from hydrogen or halogen;
m is an integer from 0 to 4;
m′ is an integer from 0 to 5;
R¹ is selected from hydrogen or a substituted or unsubstituted alkyl;
E is:

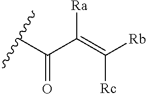

wherein Ra, Rb and Rc are independently selected from hydrogen, halogen, -CN, substituted or unsubstituted alkyl, substituted or unsubstituted heteroalkyl, substituted or unsubstituted cycloalkyl, or substituted or unsubstituted heterocyclyl; or
Ra and Rb optionally taken together with the carbon atoms to which they are attached form a 3- to 8-membered substituted or unsubstituted cycloalkyl ring, or form a 3- to 8-membered substituted or unsubstituted heterocyclyl ring; or
Rb and Rc optionally can be fused with their intervening atom to form a 3- to 8-membered substituted or unsubstituted cycloalkyl ring, or a 3- to 8- membered substituted or unsubstituted heterocyclyl ring; or
Ra and Rb optionally form a triple bond.

An embodiment of the present invention comprises compounds of Formula I or Formula II, wherein X¹ and X² are both hydrogen.

An embodiment of the present invention comprises compounds of Formula I or Formula II, wherein X¹ is fluorine and X² is hydrogen.

An embodiment of the present invention comprises compounds of Formula I or Formula II, wherein X¹ is hydrogen and X² is fluorine.

An embodiment of the present invention comprises compounds of Formula I or Formula II, wherein R¹ is hydrogen.

An embodiment includes compounds of Formula I or Formula II, wherein R¹ is selected from hydrogen or a substituted or unsubstituted C₁₋₆ alkyl.

An embodiment of the present invention comprises compounds of Formula I or Formula II, wherein R¹ is methyl.

An embodiment of the present invention comprises compounds of Formula I or Formula II, their pharmaceutically acceptable salts, solvates, solvates of salts, stereoisomers or tautomers thereof, wherein E is

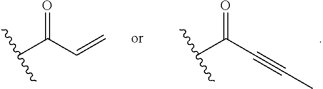

An embodiment of the present invention comprises compounds of Formula I or Formula II, wherein X¹ and X² are selected from the group consisting of hydrogen, halogen and combinations thereof;
m and m′ are an integer from 0 to 2;
R¹ is hydrogen or methyl, and

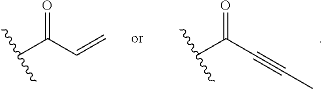

E is

An embodiment of the present invention further comprising compounds of Formula I or Formula II, wherein
X¹ and X² are selected from the group consisting of hydrogen, fluorine and combinations thereof,
m and m′ are an integer from 0 to 2;
R¹ is hydrogen or methyl, and
E is

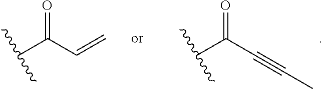

The compounds of the present invention have activity as inhibitors of protein kinases comprising members of the TEC kinase family including BTK, BLK, Tec, ITK/EMT/TSK, BMX and TXK/RLK. Most particularly, compounds of the present invention can inhibit BTK enzyme and BTK-dependent cellular functions. Additionally, compounds of the instant invention can exhibit higher selectivity for

BTK when compared to the reference compound described below, they can additionally have reduced potency against EGFR and ErbB kinases.

In an embodiment of the present invention compounds of Formula I or Formula II may be formulated into a pharmaceutical composition, which comprises an effective amount of a compound of the present invention with a pharmaceutically acceptable excipient, diluent or carrier.

According to the present invention there is provided a pharmaceutical composition which comprises a compound of Formula I or Formula II, or a pharmaceutically acceptable salt or solvate thereof, in combination with at least one pharmaceutically acceptable excipient, diluent or carrier.

Another aspect of the present invention provides compounds of Formula I or Formula II that can be administered by any means suitable for the condition to be treated, which may depend on the need for site-specific treatment or quantity of drug to be delivered. Topical administration is generally preferred for skin-related diseases, and systematic treatment preferred for cancerous or pre-cancerous conditions, although other modes of delivery are contemplated. For example, the compounds may be delivered orally, such as in the form of tablets, capsules, granules, powders, or liquid formulations including syrups; topically, such as in the form of solutions, suspensions, gels, cream or ointments; sublingually; bucally; parenterally, such as by subcutaneous, intravenous, intramuscular or intrasternal injection or infusion techniques (e.g., as sterile injectable aqueous or non-aqueous solutions or suspensions); nasally such as by inhalation spray; rectally such as in the form of suppositories; or liposomally. Dosage unit formulations containing non-toxic, pharmaceutically acceptable vehicles or diluents may be administered. The compounds may be administered in a form suitable for immediate release, extended release, delayed release or controlled release. Immediate release or extended release may be achieved with suitable pharmaceutical compositions or, particularly in the case of extended release, with devices such as subcutaneous implants or osmotic pumps. The compounds may be administered in a form suitable for targeted delivery in which the drug is only active in the target area of the body (for example, in cancerous tissues) and sustained release formulations in which the drug is released over a period of time in a controlled manner from a formulation.

The term “compound” refers also to its pharmaceutically acceptable salt, solvate, solvate of salt, stereoisomer, tautomer, isotope, prodrug, complex or biologically active metabolite thereof.

The compounds of the present invention may contain one or more acidic functional groups and, thus, are capable of forming pharmaceutically acceptable salts with pharmaceutically acceptable bases.

The term “pharmaceutically acceptable salt” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. Also salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like ((See, for example, Berge et al. (1977) “Pharmaceutical Salts”, J. Pharm. Sci. 66: 1-19).

The term “pharmaceutically effective amount” refers to any amount of the composition for the prevention and treatment of subjects that is effective in treating a disease or condition associated with protein kinase activity.

### Pharmaceutical Compositions

According to the present invention there is provided a pharmaceutical composition which comprises a compound of Formula I or Formula II, or a pharmaceutically acceptable salt or solvate thereof, in association with at least one pharmaceutically acceptable excipient, diluent or carrier.

The pharmaceutical compositions may be in a conventional pharmaceutical form suitable for oral administration (e.g., tablets, capsules, granules, powders and syrups), parenteral administration (e.g., injections (intravenous, intramuscular, or subcutaneous)), drop infusion preparations, inhalation, eye lotion, topical administration (e.g., ointment, cream), or suppositories. Regardless of the route of administration selected, the compounds may be formulated into pharmaceutically acceptable dosage forms by conventional methods known to those skilled in the art.

Compositions of the present invention intended for oral use can be prepared according to any method known to the art for the manufacture of pharmaceutical compositions, and such compositions can contain one or more agents selected from, by way of non-limiting example, sweetening agents, flavoring agents, coloring agents and preserving agents in order to provide pharmaceutically elegant and palatable preparations. Formulations suitable for oral administration can be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. Each carrier must be acceptable in the sense of being compatible with the other ingredients of the formulation, including the active ingredient, and not injurious or harmful to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. For oral formulations, “pharmaceutically acceptable carrier” such as cellulose, calcium silicate, corn starch, lactose, sucrose, dextrose, calcium phosphate, stearic acid, magnesium stearate, calcium stearate, gelatin, talc, surfactants, suspending agents, emulsifiers, diluents, and others may be used. For injectable formulations, “pharmaceutically acceptable carrier” such as water, saline, glucose solution, glucose solution analogs, alcohols, glycols, ethers (e.g., polyethylene glycol 400), oils, fatty acids, fatty acid esters, glycerides, surfactants, suspending agents, emulsifiers, and others may be used.

The term “pharmaceutically acceptable salt” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like (See, for example, Berge et al. (1977) “Pharmaceutical Salts”, J. Pharm. Sci. 66: 1-19).

The term “subject” or “patient” means a human or an animal subject for treatment.

The term “combination” within the meaning of this invention includes the simultaneous, sequential or separate use of the components or ingredients.

The pharmaceutical compositions of the present invention may be obtained by conventional procedures using conventional pharmaceutically acceptable excipients, well known in the art.

The pharmaceutical compositions of the present invention may be prepared as a sterile injectable solutions by incorporating the compounds of the present invention in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, an agent of the invention as described above may be formulated with one or more additional compounds that enhance the solubility of these agents. The invention also extends to such derivatives of such agents of the invention.

The pharmaceutical compositions of the present invention may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles, as indicated above. Alternatively, a pharmaceutical composition may be stored in a freeze-dried condition requiring only the addition of a sterile liquid carrier immediately prior to use. In one embodiment, a pharmaceutical composition is provided comprising expanded hematopoietic progenitor cells cryopreserved in a suitable cryopreservation medium, which can then be thawed and resuspended as needed for administration to a patient.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

As used herein, the term “affinity tag” means a ligand or group, linked either to a compound of the present invention or to a protein kinase domain, that allows the conjugate to be extracted from a solution.

The term “spirocycle”, as used herein, refers to bicyclic rings system connected through just one atom. The rings can be different or identical. The connecting atom, also called spiroatom, is preferably a quaternary carbon. Spirocycle may be optionally substituted with one or more substituents as defined herein.

The term “alkyl”, as used herein, refers to a saturated hydrocarbon chain. Alkyl chains may be straight or branched. Alkyl chains may be optionally substituted with one or more substituents as defined herein. Representative alkyl groups include methyl, ethyl, propyl, (n-propyl and isopropyl) butyl (n-butyl, t-butyl and isobutyl), pentyl (n-pentyl and isopentyl), hexyl and the like. In certain preferred embodiments, alkyl substituents are lower alkyl groups, e.g., having from 1 to 6 carbon atoms.

The term “alkenyl”, as used herein, refers to an unsaturated hydrocarbon chain analogous in length and possible substitution to the “alkyl” described above, but that contain at least one double bond. Representative alkenyl groups include vinyl, propen-2-yl, crotyl, isopenten-2-yl, 1,3-butadien-2-yl, 2,4-pentadienyl, and 1,4-pentadien-3-yl. In certain preferred embodiments, alkenyl substituents are lower alkenyl groups, e.g., having from 2 to 6 carbon atoms.

The term “alkynyl”, as used herein, refers to an unsaturated hydrocarbon chain analogous in length and possible substitution to the “alkyl” described above, but that contain at least one triple bond. Representative alkynyl groups include ethynyl, 1- and 3-propynyl, and 3-butynyl. In certain preferred embodiments, alkynyl substituents are lower alkyl groups, e.g., having from 2 to 6 carbon atoms.

The term, “alkylene”, as used herein, refers to an alkyl group with two open valencies.

The term “heteroalkyl”, as used herein, refers to a saturated or partially saturated chain containing one to four heteroatoms selected from the group consisting of O, N and S, and wherein the nitrogen and sulfur atoms may optionally be oxidized and the nitrogen atom may optionally be quaternized. Heteroalkyl chains may be straight or branched. Heteroalkyl chains may be optionally substituted with one or more substituents as defined herein. The heteroatom(s) O, N and S may be placed at any interior position of the heteroalkyl group. Up to two heteroatoms may be consecutive.

The term “cycloalkyl”, as used herein, alternatively “carbocycle” and “carbocyclyl” refers to a saturated or partially saturated non-aromatic ring, more preferably 3- to 8-membered ring, in which each atom of the ring is carbon or; refers to a spirocycle where each ring is a saturated or partially saturated hydrocarbon ring and the spiro atom is carbon. Cycloalkyl rings may be optionally substituted with one or more substituents as defined herein. The term “cycloalkyl”, “carbocycle” or “carbocyclyl” also include polycyclic ring systems having two or more cyclic rings in which two or more carbons are common to two adjoining rings wherein at least one of the rings is cycloalkyl, e.g., the other cyclic rings can be aryls, heteroaryls, and/or heterocyclyls. Representative cycloalkyl rings include cyclopropyl, cyclobutyl, cyclopentyl, cyclohexyl, 1-cyclohexenyl, 3- cyclohexen-1-yl, cycloheptyl, tetrahydronaphthyl, indanyl, adamantly and combinations thereof.

The term “heterocyclyl” alternatively “heterocyclic” and “heterocycloalkyl”, as used herein, refers to non-aromatic ring structures, more preferably 3- to 8-membered rings, whose ring structures include one to four heteroatoms or; refers to a spirocycle where the bicyclic rings system contains 1 to 4 heteroatoms. Heterocyclyl rings may be optionally substituted with one or more substituents as defined herein. The term “heterocyclyl” or “heterocyclic” also include polycyclic ring systems having two or more cyclic rings in which two or more carbons are common to two adjoining rings wherein at least one of the rings is heterocyclic, e.g., the other cyclic rings can be cycloalkyls, aryls and/or heteroaryls. Heterocyclyl groups include, for example, tetrahydrofuran, piperidine, piperazine, pyrrolidine, morpholine, lactones, lactams and combinations thereof.

The term “aryl”, as used herein, refers to 5-, 6-, and 7-membered aromatic rings in which each atom of the ring is carbon. Aryl rings may be optionally substituted with one or more substituents as defined herein. The term “aryl” also includes polycyclic ring systems having two or more cyclic rings in which two or more carbons are common to two adjoining rings wherein at least one of the rings is aryl, e.g., the other cyclic rings can be cycloalkyls, heteroaryls, and/or heterocyclyls. Aryl groups include, for example, benzene, naphthalene, phenanthrene, anthracene and combinations thereof.

The term “heteroaryl”, as used herein, refers to 5-, 6-, and 7-membered aromatic rings whose ring structures include one to four heteroatoms. Heteroaryl rings may be optionally substituted with one or more substituents as defined herein. The term “heteroaryl” also includes polycyclic ring systems having two or more cyclic rings in which two or more carbons are common to two adjoining rings wherein at least one of the rings is heteroaryl, e.g., the other cyclic rings can be cycloalkyls, aryls and/or heterocyclyls. Heteroaryl groups include, for example, pyrrole, furan, thiophene, imidazole, isoxazole, oxazole, thiazole, triazole, pyrazole, pyridine, pyrazine, pyridazine and pyrimidine, and combinations thereof.

The terms “polycyclyl” alternatively “polycyclic”, as used herein, refer to two or more rings (e.g., cycloalkyls, aryls, heteroaryls, and/or heterocyclyls) in which two or more carbons are common to two adjoining rings, e.g., the rings are “fused rings”. Polycyclyl rings may be optionally substituted with one or more substituents as defined herein.

The term “aralkyl”, as used herein, refers to an alkyl group substituted with an aryl group, for example —(CH₂)ₚ-Ar and p is an integer from 1 to 8 and Ar may be selected from any suitable aryl ring system, for example phenyl or napthyl. For example “aralkyl” may be benzyl.

The term “heteroaralkyl”, as used herein, refers to an alkyl group substituted with a heteroaryl group, for example —(CH₂)ₚ-Het wherein p is an integer from 1 to 8 and Het is any suitable heteroaryl ring system, such as those discussed in the above paragraphs.

The term “alkoxy”, as used herein, refers to an alkyl ether substituent, wherein the term alkyl is as defined above. Representative alkoxy groups include methoxy, ethoxy, propoxy, tert-butoxy and combinations thereof.

The term “ether”, as used herein, refers to an oxy group bridging two moieties linked at carbon atoms.

The term “alkoxyalkyl”, as used herein, refers to an alkyl group substituted with an alkoxy group, thereby forming ether.

The term “halo” or “halogen”, as used herein, refers to fluorine, chlorine, bromine and iodine.

The term “heteroatom”, as used herein, refers to an atom of any element other than carbon or hydrogen. Preferred heteroatoms are nitrogen, oxygen, and sulfur.

The term “hydrocarbon”, as used herein, refers to a group consisting entirely of carbon and hydrogen.

The term, “haloalkyl”, as used herein, refers to an alkyl substituent wherein one or more hydrogens are replaced by a halogen.

The term “carbonyl”, as used herein, when alone includes formyl —CH(O) and in combination is a —C(O) group.

The term “carboxyl”, alternatively “carboxy”, as used herein, refers to —C(O)OH or the corresponding “carboxylate” anion, such as in a carboxylic acid salt.

The term “acyl”, as used herein, refers to —C(O)R wherein R is alkyl, heteroalkyl, haloalkyl, cycloalkyl, heterocyclyl, aryl or heteroaryl as defined therein. Representative acyl groups include acetyl, trifluoroacethyl, benzoyl, and combinations thereof.

The term “alkoxycarbonyl”, as used herein, refers to —C(O)OR wherein R is alkyl as defined therein. Representative alkoxycarbonyl groups include methoxycarbonyl, ethoxycarbonyl, and the combinations thereof.

The term “alkylthio”, as used herein, refers to a thioether —SR wherein R is alkyl as defined above. Representative alkylthio groups include methylthio, ethylthio and the combinations thereof.

The term “sulfonate”, as used herein, refers to a salt or ester of a sulfonic acid —OSO₂R wherein R is alkyl, heteroalkyl, haloalkyl, cycloalkyl, heterocyclyl, aryl or heteroaryl as defined therein.

Representative sulfonate groups include mesylate, besylate, tosylate, and the combinations thereof.

The term “sulfonyl”, as used herein, refers to —SO₂R wherein R is alkyl, heteroalkyl, haloalkyl, cycloalkyl, heterocyclyl, aryl or heteroaryl as defined therein. Representative sulfonate groups include methylsufonyl, ethylsulfonyl, and the combinations thereof.

The term “sulfamoyl”, as used herein, refers to —SO₂NH₂.

The term “sulfonamido”, as used herein, refers to —S(O)₂NRR′ wherein R and R′ are independently selected from alkyl, heteroalkyl, haloalkyl, cycloalkyl, heterocyclyl, aryl or heteroaryl as defined therein. R and R′ may combine to form a heterocyclic ring.

The term “amino”, as used herein, refers to —NRR′ wherein R and R′ are independently selected from hydrogen, alkyl, heteroalkyl, haloalkyl, cycloalkyl, heterocyclyl, aryl or heteroaryl as defined therein. R and R′ may combine to form a heterocyclic ring.

The term “amido” alternatively “amide”, as used herein, refers to —C(O)NRR′ wherein R and R′ are independently slected from hydrogen, alkyl, heteroalkyl, haloalkyl, cycloalkyl, heterocyclyl, aryl or heteroaryl as defined herein. R and R′ may combine to form an heterocyclyl ring.

The term “substituted” refers to moieties having substituents replacing hydrogen on one or more atoms of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

Substituents can include, for example, an alkyl, an alkenyl, an alkynyl, a haloalkyl, a heteroalkyl, a cycloalkyl, a heterocyclyl, an aryl, a heteroaryl, a halogen, a hydroxyl, a carbonyl , carboxyl, an alkoxycarbonyl, a formyl, or an acyl, a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl. It will be understood by those skilled in the art that the substituents can themselves be substituted, if appropriate.

As used herein, the term “probe” means a compound of the invention which is labeled with either a detectable label or an affinity tag, and which is capable of binding, either covalently or non-covalently, to a protein kinase domain. When, for example, the probe is non-covalently bound, it may be displaced by a test compound. When, for example, the probe is bound covalently, it may be used to form cross-linked adducts, which may be quantified and inhibited by a test compound.

As used herein, the term “affinity tag” means a ligand or group, linked either to a compound of the present invention or to a protein kinase domain, that allows the conjugate to be extracted from a solution.

The term “prodrug” denotes a compound that is a drug precursor which, upon administration to a subject, is converted within the body into a compound of Formula I or Formula II. Prodrugs of compounds of Formula I or Formula II, or pharmaceutically acceptable salts or solvates thereof are within the scope of this disclosure.

Compounds of the invention also include all isotopes of atoms present in the Intermediates and/or final compounds. Isotopes include those atoms having the same atomic number but different mass numbers. For example, isotopes of hydrogen include deuterium and tritium.

The term “subject” or “patient” means a human or an animal subject for prevention or treatment. In an embodiment the use is ex vivo, for example in vitro, such as an in vitro assay.

The term “combination” within the meaning of this invention includes the simultaneous, sequential or separate use of the components or active pharmaceutical ingredients.

### Therapeutic Uses and Applications

The compounds of the present invention may have potential utility as inhibitors of protein kinase activity and are suitable for use in therapy.

An aspect of the present invention provides a method of inhibiting protein kinase activity in a cell, the method comprising administering to said cell compound of Formula I or Formula II as defined herein, or a pharmaceutically acceptable salt or solvate thereof.

In a further aspect, the present invention provides a method of inhibiting protein kinase in vitro or in vivo, said method comprising contacting a cell with an effective amount of a compound, or a pharmaceutically acceptable salt or solvate thereof, as defined herein.

A further aspect of the present invention provides a method of inhibiting protein kinase activity in a human or animal subject for treatment or prevention of protein kinase mediated disease, the method comprising administering to said subject an effective amount of a compound of Formula I or Formula II as defined herein, or a pharmaceutically acceptable salt or solvate thereof.

The term “protein kinase mediated disease” is used herein associated with abnormal or undesirable cellular responses triggered by protein kinase-mediated events. Furthermore, aberrant activation or excessive expressions of various protein kinases are implicated in the mechanism of multiple diseases and disorders. These diseases include, but are not limited to allergies and asthma, Alzheimer's disease, autoimmune diseases, bone diseases, cancer, cardiovascular diseases, inflammatory diseases, hormone-related diseases, metabolic diseases, neurological and neurodegenerative diseases. Thus, inhibitors of kinase families are expected to be suitable in the treatment of cancer, vascular disease, autoimmune diseases, and inflammatory conditions including, but not limited to: solid tumors, hematological malignancies, thrombus, arthritis, graft versus host disease, lupus erythematosus, psoriasis, colitis, illeitis, multiple sclerosis, uveitis, coronary artery vasculopathy, systemic sclerosis, pemphigus, atherosclerosis, asthma, transplant rejection, allergy, dermatomyositis and other conditions such as stroke, gout, type 2 diabetes, obesity-induced insulin resistance, atherosclerosis and Muckle-Wells syndrome.

In one embodiment, the protein kinase inhibited by compounds of the present invention is BTK.

The compounds of the present invention may be used in the treatment or prevention of diseases that involve BTK, i.e. diseases that involve B cells and/or mast cells, for example, cancer, autoimmune diseases, allergic diseases, inflammatory diseases, graft-versus-host disease, thromboembolic diseases, bone-related diseases, infectious diseases, viral infections and the like. Examples of cancer in the present invention include non-Hodgkin's lymphomas, for example, Burkitt's, lymphoma, AIDS-related lymphoma, marginal zone B-cell lymphoma (nodal marginal zone B cell lymphoma, extranodal marginal zone B-cell lymphoma, splenic marginal zone B-cell lymphoma), diffuse large B-cell lymphoma, primary effusion lymphoma, lymphoma-like granulomatous disease, follicular lymphoma, B-cell chronic lymphocytic leukemia, B cell prolymphocytic leukemia, lymphoplasmacytic leukemia/Waldenstrom's macroglobulinemia, plasmacytoma, mantle cell lymphoma, mediastinal large B-cell lymphoma, intravascular large B-cell lymphoma, and hairy cell leukemia. Moreover, examples of cancer in the present invention include cancers other than non-Hodgkin's lymphoma such as pancreatic endocrine tumors and multiple myeloma. Examples of pancreatic endocrine tumors include insulinoma, gastrinoma, glucagonoma, somatostatinoma, VIP-producing tumor, PP-producing tumor, GRF-producing tumor, and the like. Examples of an autoimmune disease in the present invention include but not limiting to inflammatory bowel disease, arthritis, rheumatoid arthritis, psoriatic arthritis, osteoarthritis, Still's disease, juvenile arthritis, type I diabetes, myasthenia gravis, Hashimoto's thyroiditis, Ord's thyroiditis, Basedow's disease, Sjogren's syndrome, multiple sclerosis, Guillain-Barre syndrome, acute disseminated encephalomyelitis, Addison disease, opsoclonus-myoclonus syndrome, ankylosing spondylitis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's disease, Takayasu arteritis, temporal arteritis, warm autoimmune hemolytic anemia, Wegener granuloma, psoriasis, alopecia universalis, Burchett disease, chronic fatigue syndrome, dysautonomia, endometriosis, interstitial cystitis, myotonia, vulvodynia, pemphigus, systemic lupus erythematosus, and the like.

Examples of an allergic disease in the present invention include allergy, anaphylaxis, allergic conjunctivitis, allergic rhinitis, atopic dermatitis and the like.

Examples of an inflammatory disease in the present invention include asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, epicondylitis, epididymitis, fasciitis, fibrositis, gastritis, gastroenteritis, hepatitis, hidradenitis suppurativa, laryngitis, mastitis, meningitis, myelitis, myocarditis, myositis nephritis, oophoritis, orchitis, osteitis, pancreatitis, parotitis, pericarditis, peritonitis, pharyngitis, pleuritis, phlebitis, pneumonia, proctitis, prostatitis, pyelonephritis, rhinitis, salpingitis, sinusitis, stomatitis, synovitis, tendinitis, tonsillitis, uveitis, vaginitis, vasculitis, vulvitis, and the like.

Examples of a thromboembolic disease in the present invention include myocardial infarction, angina pectoris, reocclusion after angioplasty, restenosis after angioplasty, reocclusion after aortocoronary bypass, restenosis after aortocoronary bypass, cerebral infarction, transient ischemia, peripheral vascular occlusive disease, pulmonary embolism, deep vein thrombosis, and the like.

Examples of a bone-related disease in the present invention include osteoporosis, periodontitis, metastasis of cancer to bone, osteoarthritis, hypercalcemia, bone fractures, and the like.

Examples of a viral infection in the present invention include HIV infection.

In one embodiment, the compound of Formula I or Formula II or pharmaceutically acceptable salts, solvates, solvates of salts, stereoisomers, tautomers, isotopes, prodrugs, complexes, or biologically active metabolites thereof, is acting by inhibiting one or more of the host cell kinases involved in cell proliferation, cell survival, viral production, cardiovascular disorders, neurodegeneration, autoimmunity, a metabolic disorder, stroke, alopecia, an inflammatory disease or an infectious disease.

The compounds object of the present invention may be administered 1 to 4 times a day. A dosage may be between 0.01-100 mg/kg body weight/day of the compound object of the present invention may be administered to a patient receiving these compositions. The dose can vary within wide limits and is to be suited to the individual conditions in each individual case. For the above uses the appropriate dosage will vary depending on the mode of administration, the particular condition to be treated and the effect desired. Preferably a dose of 1 to 50 mg/kg body weight/day may be used.

In an embodiment of the present invention suitable dosage rates for a subject, for example humans, are of the order of from about 10 mg to 3 g/day, administered orally once, or divided doses, such as 2 to 4 times a day, or in sustained release form. For topical delivery, depending on the permeability of the skin, the type and the severity of the disease and dependent on the type of formulation and frequency of application, different concentrations of active compounds within the medicament can be sufficient to elicit a therapeutic effect by topical application. Preferably, the concentration of an active compound pharmaceutically acceptable salts, solvates, solvates of salts, stereoisomers, tautomers, isotopes, prodrugs, complexes or biologically active metabolites thereof, within a medicament according to the present invention is in the range of between 1 μmol/L and 100 mmol/L.

In further aspect of the present invention, the compound of Formula I or Formula II, or pharmaceutically acceptable salts, solvates, solvates of salts, stereoisomers, tautomers, isotopes, prodrugs, complexes, or biologically active metabolites thereof, act as inhibitors of cell kinases as anti-inflammatory, anti-cancer, anti-viral and as antithrombotic agents.

The compounds and/or pharmaceutically acceptable salts of the present invention may be used in combination with one or more other drugs in the treatment of diseases or conditions for which compounds of the present disclosure or the other drugs may have utility, where the combination of the drugs together are safer or more effective than either drug alone. Such other drug(s) may be administered, by a route and in an amount commonly used therefore, contemporaneously or sequentially with a compound of the present disclosure. When a compound and/or pharmaceutically acceptable salt of the present disclosure is used contemporaneously with one or more other drugs, a pharmaceutical composition in unit dosage form containing such other drugs and the compound and/or pharmaceutically acceptable salt of the present disclosure is preferred. However, the combination therapy may also include therapies in which the compound and/or pharmaceutically acceptable salt of the present disclosure and one or more other drugs are administered on different overlapping schedules. It is also contemplated that when used in combination with one or more other active ingredients, the compounds and/or pharmaceutically acceptable salts of the present disclosure and the other active ingredients may be used in lower doses than when each is used singly. Accordingly, the pharmaceutical compositions of the present disclosure also include those that contain one or more other active ingredients, in addition to a compound and/or pharmaceutically acceptable salt of the present disclosure.

The above combinations include combinations of a compound of the present disclosure not only with one other active compound, but also with two or more other active compounds. Likewise, compounds and/or pharmaceutically acceptable salts of the present disclosure may be used in combination with other drugs that are used in the prevention, treatment, control, amelioration, or reduction of risk of the diseases or conditions for which compounds of the present disclosure are useful. Such other drugs may be administered, by a route and in an amount commonly used therefore by those skilled in the art, contemporaneously or sequentially with a compound and/or pharmaceutically acceptable salt of the present disclosure. When a compound and/or pharmaceutically acceptable salt of the present disclosure is used contemporaneously with one or more other drugs, a pharmaceutical composition containing such other drugs in addition to the compound and/or pharmaceutically acceptable salt of the present disclosure is preferred. Accordingly, the pharmaceutical compositions of the present disclosure also include those that also contain one or more other active ingredients, in addition to a compound and/or pharmaceutically acceptable salt of the present disclosure. The weight ratio of the compound and/or pharmaceutically acceptable salt of the present disclosure to the second active ingredient may be varied and will depend upon the effective dose of each ingredient. Generally, an effective dose of each will be used.

Where the patient is suffering from or at risk of suffering from an autoimmune disease, an inflammatory disease, or an allergy disease, a compound and/or pharmaceutically acceptable salt of present disclosure can be used in with one or more of the following therapeutic agents in any combination: immunosuppressants (e.g., tacrolimus, diethylstilbestrol, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non- steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, allergy vaccines, antihistamines, antileukotrienes, beta-agonists, theophylline, and anticholinergics.

Where the patient is suffering from or at risk of suffering from a B-cell proliferative disorder (e.g., CLL and SLL) the patient can be treated with a compound and/or pharmaceutically acceptable salt disclosed herein in any combination with one or more other anti-cancer agents. Examples of anti-cancer agents include, but are not limited to, any of the following: fludarabine, cladribine, chlorambucil, cyclophosphamide, vincristine, doxorubicin, mitoxantrone, bendamustine and prednisone.

Additionally, where the patient is suffering from or at risk of suffering from a cancer, autoimmune, inflammation or other disease such as HIV the patient can be treated with a compound and/or pharmaceutically acceptable salt disclosed herein in any combination with one or more checkpoint inhibitors including but not limited to PD-1 or PDL-1 antibodies such as: pembrolizumab, nivolumab, pidilizumab, BMS 936559, MPDL328OA and fragments, derivatives, conjugates, variants, radioisotope-labeled complexes and biosimilars thereof. Other agents that can be used in combination with a compound and/or pharmaceutically acceptable salt of present disclosure include anti-CD20 antibodies (including rituximab, obinutuzumab, ofatumumab, veltuzumab, tositumomab, ibritumomab), TNF inhibitors (including: infliximab, adalimumab, certolizumab pegol, golimumab, and etanercept), IL-6 inhibitors (including: tocilizumab, siltuximab, sarilumab, olokizumab, elsilimomab and sirukumab), IL-1lbeta inhibitors (including: canakinumab and Anakinra), interferons (including; Interferon alpha 2a, Interferon alpha 2b, Interferon beta 1a, Interferon beta 1b, Interferon gamma 1b, PEGylated interferon alpha 2a, and PEGylated interferon alpha 2b) and fragments, derivatives, conjugates, variants, radioisotope-labeled complexes and biosimilars thereof.

In some circumstances the patient can be treated with a compound and/or pharmaceutically acceptable salt disclosed herein in any combination with one or more anticoagulant or antiplatelet active pharmaceutical ingredients including but not limited to: acenocoumarol, anagrelide, abciximab, aloxiprin, antithrombin, apixaban, argatroban, asprin, asprin with extended release dipyridamole, beraprost, betrixaban, bivalirudin, carbasalate calcium, cilostaxol, clopidogrel, glopidogrel bisulfate, bloricromen, dabigatran etexilate, darexaban, dalteparin, dalteparin sodium, defibrotide, dicumarol, diphenadione, dipyridamole, ditaxole, desirudin, edoxaban, enoxaparin, enoxaparin sodium, epitifibatide, fondaparinux, fondaparinux sdium, heparin, heparin sodium, heparin calcium, idraparinux, idraparinux sodium, iloprost, indobufen, lepirudin, low molecular weight heparin, melagatran, nadroparin, otamixaban, parnaparin, phenindione, pheprocoumon, parsugrel, picotamide, prostacyclin, ramatroban, reviparin, rivaoxaban, sulodexide, terutroban, terutroban sodium, tricgrelor, ticlopidine, ticlopidine hydrochloride, tinzapaprin, tinzaparin sodium, tirofiban, tirfiban hydrochloride, treprostinil, treprostinil sodium, triflusal, vorapaxar, warfarin, warfarin sodium, ximelagatran, salts thereof, solvates thereof, hydrates thereof and combinations thereof.

As defined herein an effect against a proliferative disorder mediated by a kinase within the scope of the present invention may be demonstrated by the ability to inhibit a purified kinase in vitro or to inhibit cell proliferation or survival in an in vitro cell assay, for example in BTK Kinase Inhibition Assay and Splenic Cell Proliferation Assay. These assays are described in more details in the accompanying examples.

The present invention contemplates compounds of Formula I or Formula II or pharmaceutical salts thereof. The invention also contemplates solvates, solvates of salts, stereoisomers, tautomers, isotopes, prodrugs, complexes or biologically active metabolites of the compounds of Formula I or Formula II.

### Specific Abbreviations Used

ABL Abelson Murine Leukemia viral oncogene homolog

ACK Cytoplasmic Tyrosine Kinases

AIDS Acquired Immune Deficiency Syndrome

ATP Adenosine Triphosphate

BMX/ETK Bone Marrow-expressed Kinase

Boc₂O Di-tert-butyl dicarbonate

BTK Bruton's Tyrosine Kinase

BMS 936559 Bristol-Myers Squibb

CLL Chronic lymphocytic leukemia

CSK Tyrosine-protein kinase (C-src tyrosine kinase)

CD20 B-lymphocyte antigen is an activated-glycosylated phosphoprotein

Cul Copper (I) Iodide

CuCl₂ Copper(II) Chloride

Cs₂CO₃ Cesium Carbonate

DIAD Diisopropyl Azodicarboxylate

DME Ethylene Glycol Dimethyl Ether

DMF Dimethylformamide

EGFR Epidermal Growth Factor Receptor

Erbb Family of proteins contains four Receptor Tyrosine Kinases related to Epidermal Growth Factor Receptor

EPH Erythropoietin-Producing Hepatocellular

FER Proto-oncogene Tyrosine Protein Kinase

FAK Focal Adhesion Kinase

FGFR Fibroblast Growth Factor Receptor

FTY720 2-Amino-2-[2-(4-octyl-phenyl)-ethyl]-propane-1,3-diol hydrochloride, Fingolimod hydrochloride

GRF Growth Hormone Releasing Factor

H₂ Hydrogen

HCl Hydrogen Chloride

HATU (1-[Bis(dimethylamino)methylene]-1H-1,2,3-triazolo[4,5-b]pyridinium3-oxid hexafluorophosphate)

HIV Human Immunodeficiency Virus

JAK Janus Kinase

IL-6 Interleukin 6 inhibitors

ITK Inducible T-cell Kinase

K₂CO₃ Potassium Carbonate

LiAlH₄ Lithium Aluminum Hydride

μl microliter

ml milliliter

MS mass spectrometry

mmol millimole

MgSO4 Magnesium Sulfate

NaOH Sodium Hydroxide

Na₂SO₃ Sodium Sulfite

NaHCO₃ Sodium Bicarbonate

NH₄OH Ammonium Hydroxide

NIS N-iodosuccinimide

NMP N-methyl-2-pyrrolidone

Pd/C Palladium on Carbon

PDL-1 Programmed Death-Ligand 1

PD-1 Programmed Cell Death Protein 1

Ph₃P Triphenyl Phosphine

PdCl₂(dppf) [1,1′-Bis(diphenylphosphino)ferrocene]dichloropalladium(II)

Pd(OAc)₂ Palladium (II) Acetate

RLK/TXK Resting Lymphocyte Kinase

RET Proto-Oncogene

SLL Small Lymphocytic Lymphoma

SRC Proto-Oncogene Encoding a Tyrosine Kinase Syk

TEA Triethylamine

TEC Tyrosine-protein Kinase

THF Tetrahydrofuran

VEGFR Vascular Endothelial Growth Factor Receptor

VIP-producing tumor Vasoactive Intestinal Peptide-Producing Tumor

XPhos 2-Dicyclohexylphosphino-2′,4′,6′-triisopropylbiphenyl

### General Synthetic Methods

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

The following section describes general synthetic method(s) which may be useful in the preparation of compounds of the instant invention.

Compounds of Formula I or Formula II were prepared from commercially available starting materials as shown in Schemes A, B, B′, C, D and E.

Intermediates A3, B6 and B10 were prepared from commercially available starting material as shown in Schemes A, B and B′.

Intermediate A3 is obtained in a 2 steps sequence starting from commercially available starting material A1. Amination of Intermediate Al provides Intermediate A2, halogenation of Intermediate A2 provides Intermediate A3.

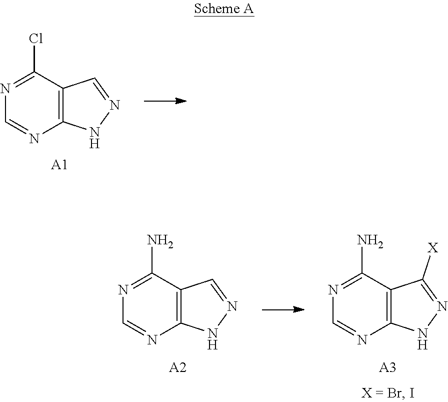

Addition of Intermediate B1 to commercially available nitro derivative B2 in a presence of a base provides Intermediate B3. Reduction of the nitro group to the corresponding amine provides Intermediate B4. Substitution of the amino group via preparation of its diazonium salt and subsequent displacement provide halogen Intermediate B5. A metal-catalysed cross coupling reaction of halogen Intermediate B5 with a tetraalkoxydiboron or dialkoxyhydroborane provides arylboronates Intermediates of formula B6 (Ra′ and Rb′ are C₁-C₆ alkyl or Ra′ and Rb′ combine to form a cyclic boronic ester), the corresponding aryl boronic acids can be further obtained by hydrolysis (Ra′ and Rb′ are hydrogen).

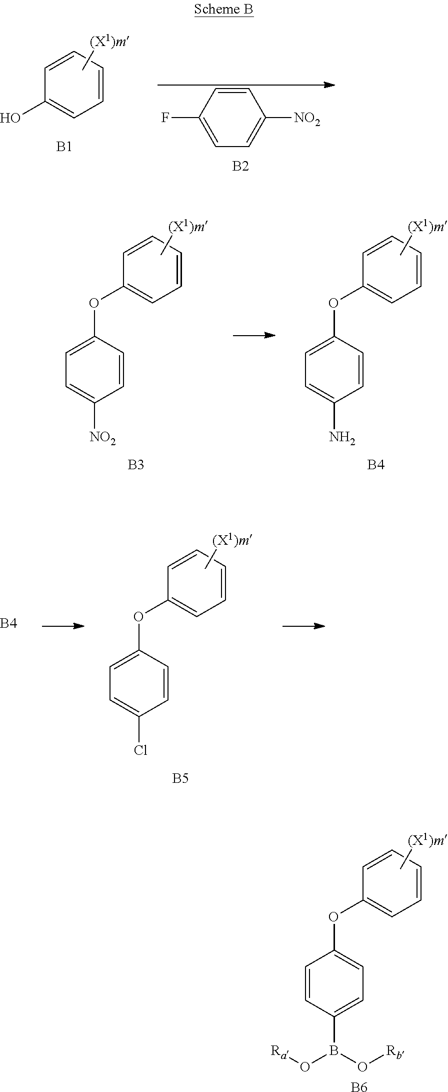

An Ullmann cross coupling reaction between Intermediate B7 and commercially available aryl bromide derivative B8 provides Intermediate B9. A metal-catalysed cross coupling reaction of halogen Intermediate B9 with a tetraalkoxydiboron or dialkoxyhydroborane provides arylboronates Intermediates of formula B10 (Ra′ and Rb′ are C₁-C₆ alkyl or Ra′ and Rb′ combine to form a cyclic boronic ester), the corresponding aryl boronic acids can be further obtained by hydrolysis (Ra′ and Rb′ are hydrogen).

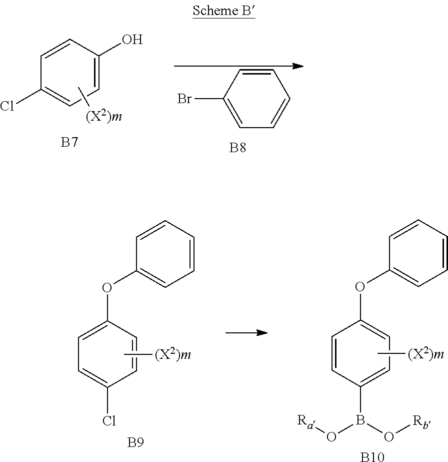

Compounds of Formula I were prepared from Intermediates A3, B6, B10 and commercially available starting materials as shown in Schemes C, D and E.

Intermediate C1 is coupled to Intermediate A3 via Mitsunobu reaction to give Intermediate C2. P is an appropriate amine protective group.

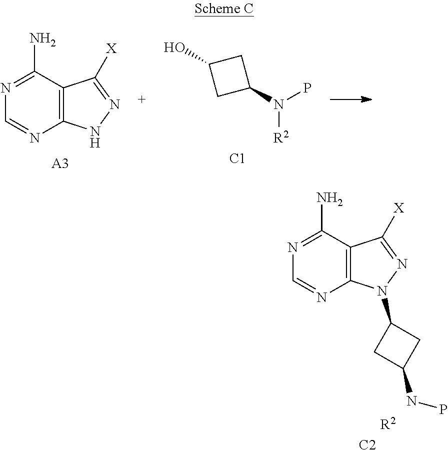

Metal catalyst cross coupling reaction of Intermediate of formula C2 with a boronic acid or boronate ester of formula B6 or B10 under Suzuki coupling reaction conditions provide Intermediate Dl. Deprotection of Intermediate D1 provides Intermediate D2.

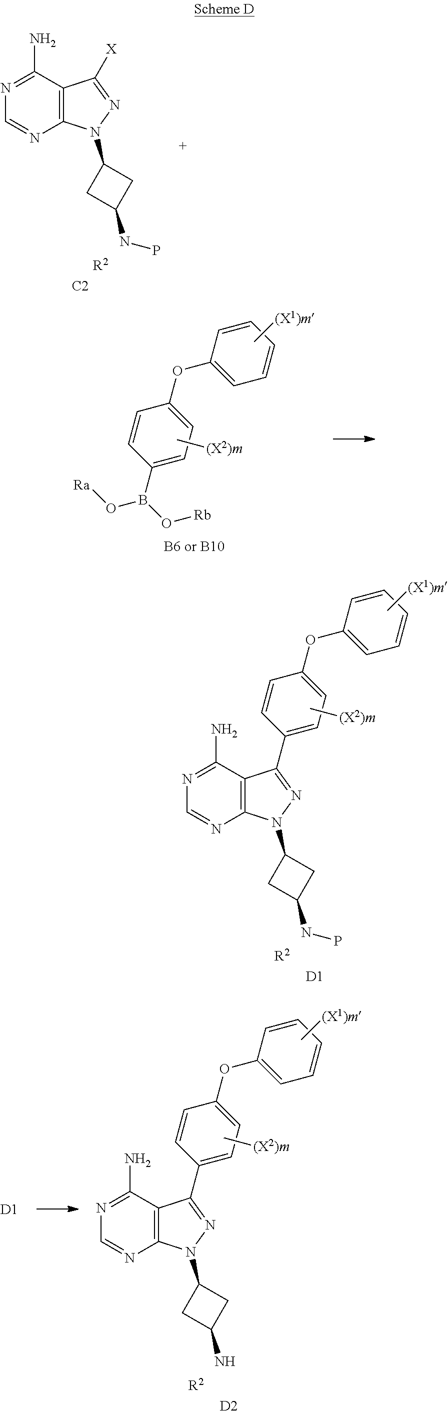

Compounds of Formula I are obtained from Intermediate D2 by acylation.

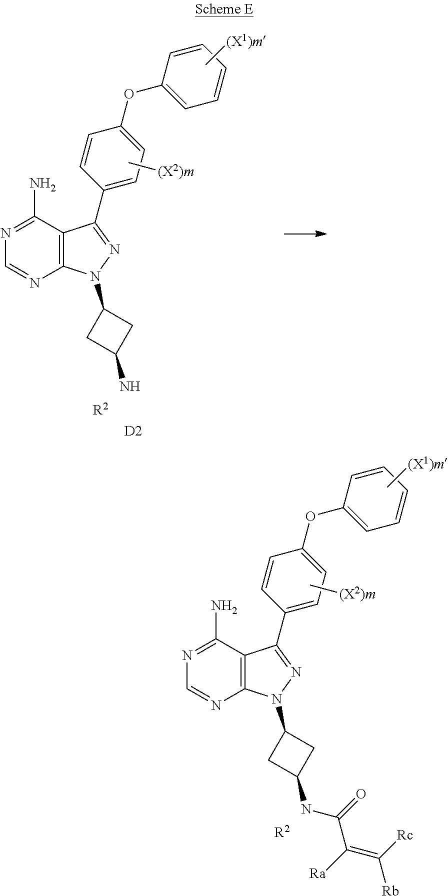

The following synthetic methods are intended to be representative of the chemistry used to prepare compounds of Formula I or Formula II of the present invention, and are not intended to be limiting.

### Synthesis of Intermediate 1-c:

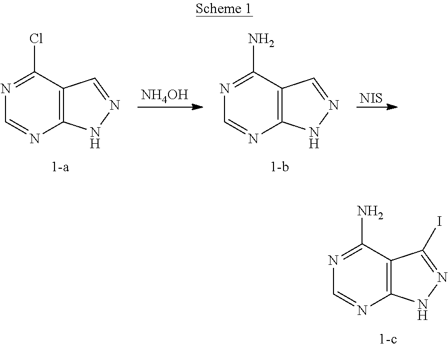

### Step 1: Intermediate 1-b

To a solution of Intermediate 1-a (20.0 g, 129.0 mmol) in 2-propanol (90 ml) was added ammonium hydroxide (126 ml). The reaction was heated in a pressure vessel at 95° C. overnight then cooled to room temperature. Volatiles were removed under reduced pressure. The residue was triturated in water; a precipitate formed and was collected by filtration to provide Intermediate 1-b as a white solid.

### Step 2: Intermediate 1-c

To a solution of Intermediate 1-b (14.2 g, 105.0 mmol) in DMF (120 ml) was added N-iodosuccinimide (35.5 g, 158.0 mmol) and the reaction was heated at 55° C. overnight and then cooled to room temperature. A saturated aqueous solution of Na₂SO₃ was added; a precipitate formed and was collected by filtration, washed with a saturated aqueous solution of Na₂SO₃ and then dried under vacuum to provide Intermediate 1-c as an off-white solid.

### Synthesis of Intermediates 2-b and 2-d:

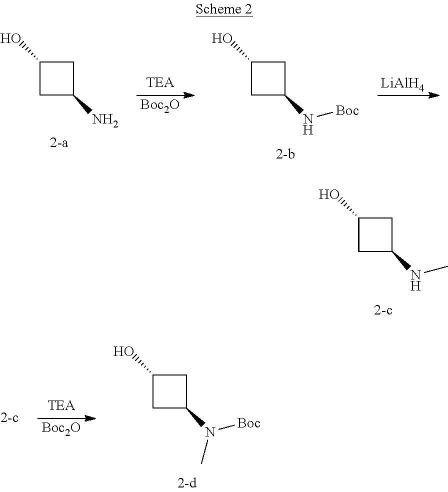

### Step 1: Intermediate 2-b

To a solution of in Intermediate 2-a·HCl (10.0 g, 70.6 mmol) in ethanol (35 ml) were sequentially added TEA (35.0 ml) and Boc₂O (20.0 g, 31.3 mmol) and the reaction was stirred overnight at room temperature. Volatiles were removed under reduced pressure, ethyl acetate and water were added to the residue, the organic layer was separated, washed with a saturated aqueous solution of NaHCO₃ and brine, dried over MgSO₄, filtered and concentrated under reduced pressure to provide Intermediate 2-b as a white solid.

### Step 2: Intermediate 2-c

To a solution of Intermediate 2-b (2.0 g, 10.7 mmol) in anhydrous THF (53 ml) cooled to 0° C. was slowly added a 1.0 M solution of LiALH₄ in THF (32.0 ml, 31.0 mmol). After the addition was completed, the reaction was warmed to room temperature, stirred at 65° C. for 2 hours and then cooled to 0° C. 15% aqueous NaOH was then added and after stirring for 15 minutes the reaction was filtered. The filtrate was concentrated under reduced pressure. Purification by silica gel chromatography provided Intermediate 2-c as a white solid.

Step 3: Intermediate 2-d To a solution of in Intermediate 2-c (800 mg, 7.9 mmol) in ethanol (5.0 ml) were sequentially added TEA (4.9 ml) and Boc₂O (1.7 g, 7.9 mmol) and the reaction was stirred for 4 days at room temperature. Volatiles were removed under reduced pressure, dichloromethane and water were added to the residue, the organic layer was separated, the aqueous layer was extracted twice with dichloromethane, the combined organic extracts were washed with a saturated aqueous solution of NaHCO₃ and brine, dried over MgSO₄, filtered and concentrated under reduced pressure to provide Intermediate 2-d as a colorless oil.

### Synthesis of Intermediate 3-a:

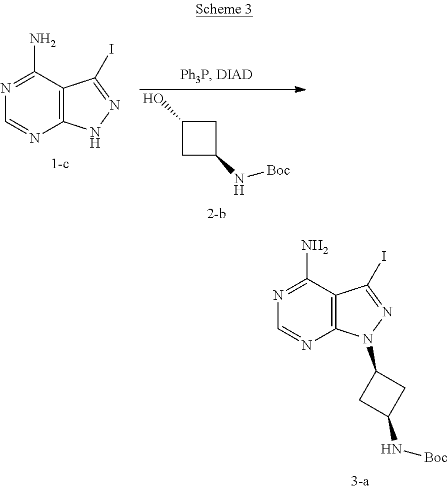

To a solution of Intermediate 2-b (3.9 g, 21.1 mmol) and triphenylphosphine (6.5 g, 24.9 mmol) in THF cooled to 0° C. was added DIAD (4.8 ml, 24.9 mmol). After the addition was completed, Intermediate 1-c (5.0 g, 19.2 mmol) was added and the reaction was slowly warmed to room temperature and stirred overnight. Volatiles were removed under reduced pressure and the residue was adsorbed on silica gel. Purification by silica gel chromatography provided Intermediate 3-a as a white solid.

### Synthesis of Intermediate 4-a:

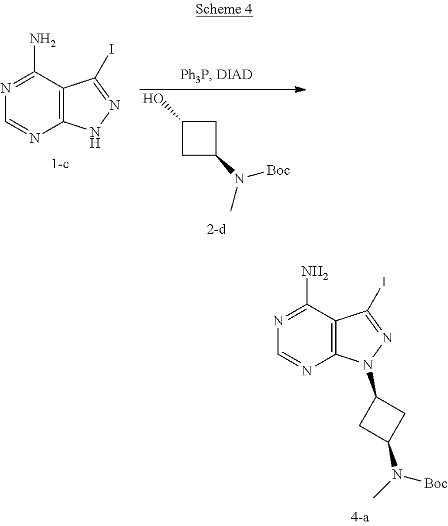

To a solution of Intermediate 2-d (763 mg, 3.8 mmol) and triphenylphosphine (1.2 g, 4.5 mmol) in THF cooled to 0° C. was added DIAD (872 μl, 4.5 mmol). After the addition was completed, Intermediate 1-c (900 mg, 3.4 mmol) was added and the reaction was slowly warmed to room temperature and stirred overnight. Volatiles were removed under reduced pressure and the residue was adsorbed on silica gel. Purification by silica gel chromatography provided Intermediate 4-a as a yellow solid.

### Synthesis of Intermediate 5-f:

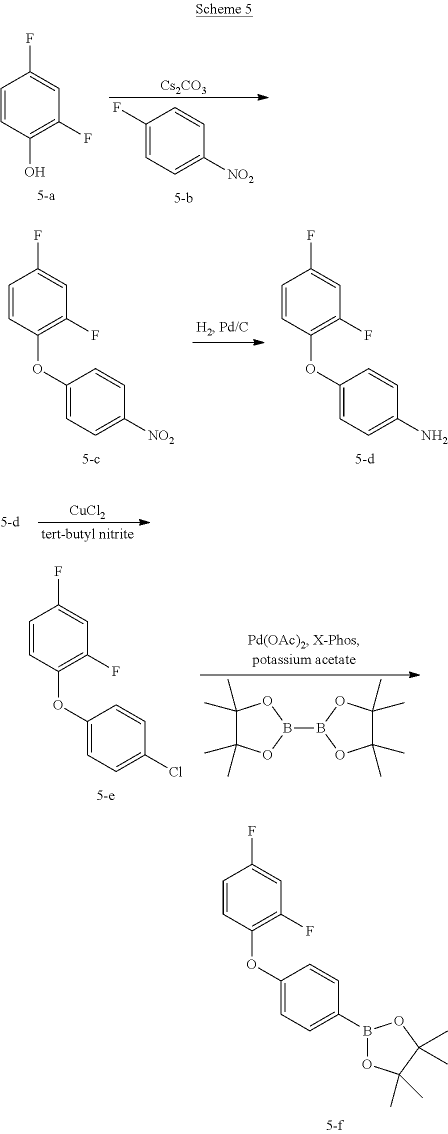

### Step 1: Intermediate 5-c

A solution of 1-fluoro-4-nitrobenzene (1.0 g, 7.1 mmol) and 2,4-difluorophenol (922 mg, 7.1 mmol) and cesium carbonate (4.6 g, 14.2 mmol) in NMP (35.4 ml) was stirred at 100° for 2 hours and then cooled to room temperature. Water was added; a precipitate formed and was collected by filtration, washed with water and then dried under vacuum to provide Intermediate 5-c as a yellow solid.

### Step 2: Intermediate 5-d

To a solution of Intermediate 5-c (1.4 g, 5.6 mmol) in methanol was added palladium on carbon (593 mg, 0.3 mmol) and the suspension was stirred for 1 hour under 60 psi of hydrogen. The reaction was filtered over celite, volatiles were removed under reduced pressure to provide Intermediate 5-d as a colorless oil.

### Step 3: Intermediate 5-e

To a solution of Intermediate 5-d (1.2 g, 5.4 mmol) and copper (II) chloride (1.1 g, 8.1 mmol) in acetonitrile (36.2 ml) was added tert-butyl nitrite (615 mg, 6.0 mmol). After the addition was completed, the reaction was heated for 2 hours and then cooled to room temperature. A saturated aqueous solution of ammonium chloride and ethyl acetate were added, the organic layer was separated, washed with brine, dried over MgSO₄, filtered and concentrated under reduced pressure. Purification by silica gel chromatography provided Intermediate 5-e as a beige solid.

### Step 4: Intermediate 5-f

A degassed solution of Intermediate 5-e (800 mg, 3.3 mmol), Bis(pinacolato)diboron (929 mg, 3.7 mmol), Palladium(II) acetate (37 mg, 0.17 mmol), potassium acetate (979 mg, 0.17 mmol) and X-Phos (158 mg, 0.33 mmol) in 1,4-dioxane (6.6 ml) was heated in a pressure vessel at 110° C. overnight and then cooled to room temperature. A saturated aqueous solution of ammonium chloride and ethyl acetate were added, the organic layer was separated, washed with brine, dried over MgSO₄, filtered and concentrated under reduced pressure. Purification by silica gel chromatography provided Intermediate 5-f as a yellow solid.

### Synthesis of Intermediate 6-d:

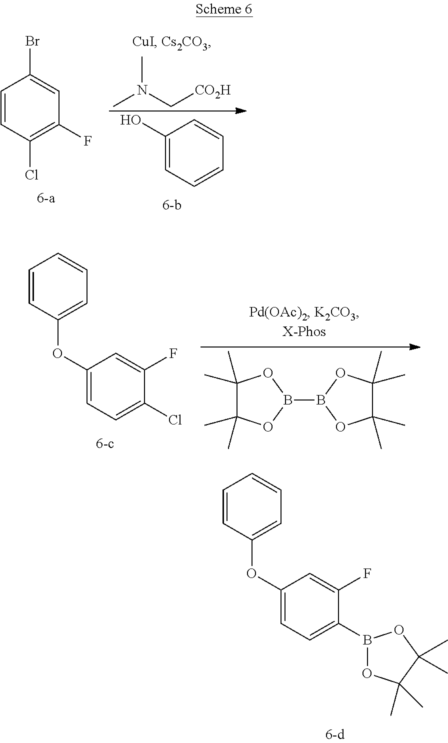

### Step 1: Intermediate 6-c

A suspension of 1-chloro-2-fluoro-4-iodobenzene (2.9 g, 11.2 mmol), phenol (1.0 g, 10.7 mmol), N,N-Dimethylglycine (3.3 g, 31.9 mmol), cesium carbonate (17.3 g, 53.1 mmol) and copper(I) iodide (2.0 g, 10.6 mmol) in 1,4-dioxane (30 ml) was heated at 110° C. overnight and then cooled to room temperature. Ethyl acetate was added, the reaction was filtered over celite and the filtrate was concentrated under reduced pressure. Purification by silica gel chromatography provided Intermediate 6-c as a colorless oil.

### Step 2: Intermediate 6-d

A degassed solution of Intermediate 6-c (1.7 g, 7.6 mmol), Bis(pinacolato)diboron (2.1 g, 8.4 mmol), Palladium(II) acetate (86 mg, 0.4 mmol), potassium acetate (2.3 g, 22.9 mmol) and X-Phos (364 mg, 0.8 mmol) in 1,4-dioxane (15 ml) was heated in a pressure vessel at 110° C. overnight and then cooled to room temperature. A saturated aqueous solution of ammonium chloride and ethyl acetate were added, the organic layer was separated, washed with brine, dried over MgSO₄, filtered and concentrated under reduced pressure. Purification by silica gel chromatography provided Intermediate 6-d as a yellow solid.

### Synthesis of Intermediate 7-b:

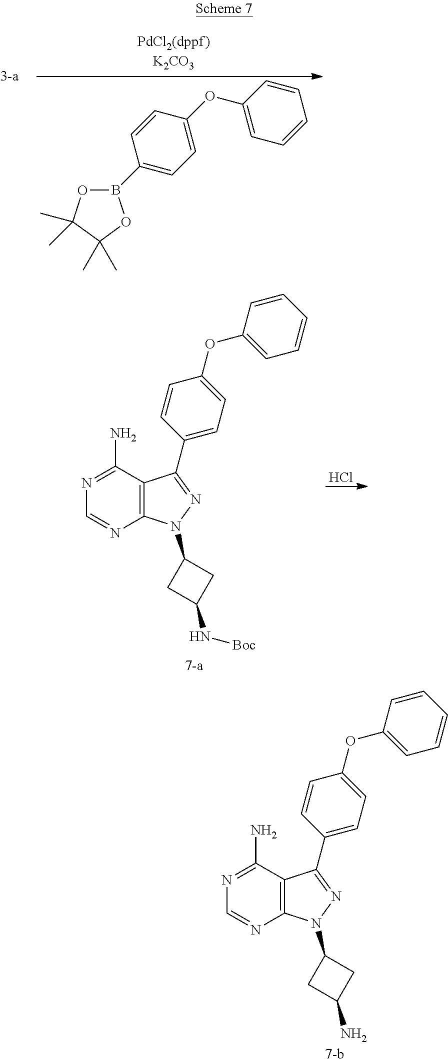

### Step 1: Intermediate 7-a

To a degassed solution of Intermediate 3-a (3.0 g, 7.8 mmol), 4,4,5,5-tetramethyl-2-(4-phenoxyphenyl)-1,3,2-dioxaborolane (2.4 g, 8.2 mmol) and potassium carbonate (3.2 g, 23.5 mmol) in DME (41.7 ml) and water (10.4 ml) was added PdCl₂(dppf) (573 mg, 0.8 mmol) and the reaction was heated in a pressure vessel at 105° C. for 3 hours and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided Intermediate 7-a as a white solid.

### Step 2: Intermediate 7-b

To a solution of Intermediate 7-a (2.3 g, 4.8 mmol) in 1,4-dioxane (10 ml) and methanol (1 ml) cooled to 0° C. was added a solution of 4N HCl in 1,4-dioxane (10.0 ml, 40.0 mmol). After the addition was completed the reaction was stirred for 3 hours at room temperature. THF was added, a precipitate formed and was collected by filtration to provide Intermediate 7-b·2HCl as an off-white solid.

### Synthesis of Intermediate 8-b:

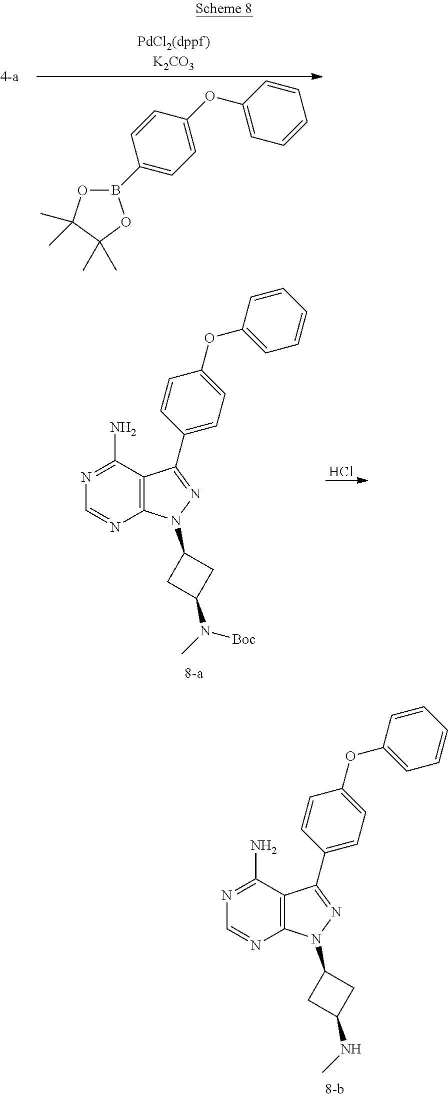

### Step 1: Intermediate 8-a

To a degassed solution of Intermediate 4-a (3.0 g, 7.8 mmol), 4,4,5,5-tetramethyl-2-(4-phenoxyphenyl)-1,3,2-dioxaborolane (328 mg, 1.1 mmol) and potassium carbonate (306 mg, 2.2 mmol) in DME (3.9 ml) and water (1.0 ml) was added PdCl₂(dppf) (54 mg, 0.07 mmol) and the reaction was heated in a pressure vessel at 105° C. overnight and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided Intermediate 8-a as a white solid.

### Step 2: Intermediate 8-b

To a solution of Intermediate 8-a (359 mg, 0.7 mmol) in 1,4-dioxane (10 ml) and methanol (1 ml) cooled to 0° C. was added a solution of 4N HCl in 1,4-dioxane (3.7 ml, 14.7 mmol). After the addition was completed the reaction was stirred for 1 hour at 0° C. Volatiles were removed under reduced pressure, Purification by reverse phase chromatography provided Intermediate 8-b·2HCl as a white solid.

### Synthesis of Intermediate 9-b:

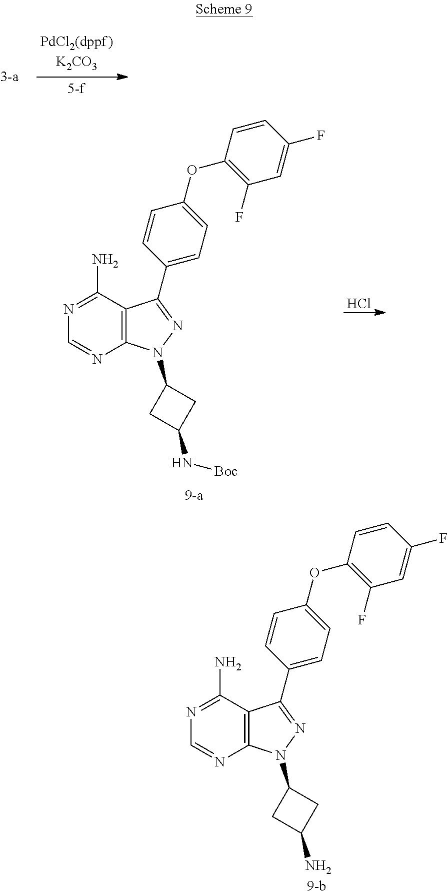

### Step 1: Intermediate 9-a

To a degassed solution of Intermediate 3-a (340 mg, 0.8 mmol), Intermediate 5-f (276 mg, 0.8 mmol) and potassium carbonate (328 mg, 2.4 mmol) in DME (4.2 ml) and water (1.0 ml) was added PdCl₂(dppf) (58 mg, 0.08 mmol) and the reaction was heated in a pressure vessel at 105° C. overnight and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided Intermediate 9-a as a beige solid.

### Step 2: Intermediate 9-b

To a solution of Intermediate 9-a (400 mg, 0.8 mmol) in 1,4-dioxane (10 ml) and methanol (1 ml) cooled to 0° C. was added a solution of 4N HCl in 1,4-dioxane (10 ml, 40 mmol). After the addition was completed the reaction was stirred for 30 minutes at room temperature. Diethyl ether was added, a precipitate formed and was collected by filtration to provide Intermediate 9-b·2HCl as a white solid.

### Synthesis of Intermediate 10-b:

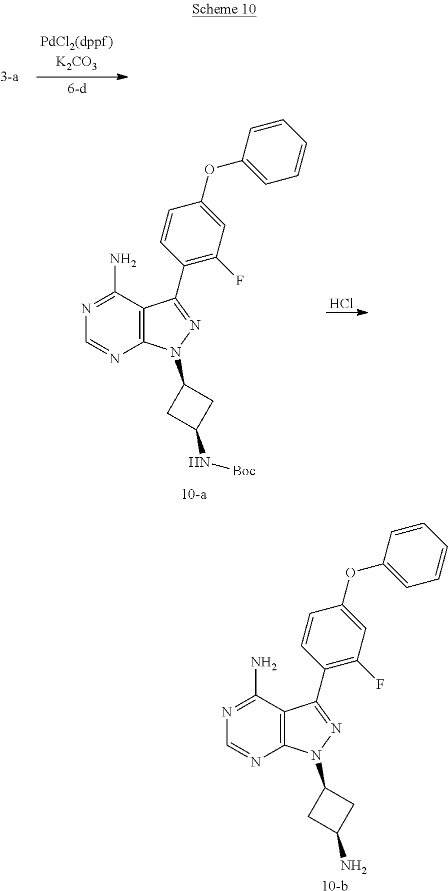

### Step 1: Intermediate 10-a

To a degassed solution of Intermediate 3-a (1.0 g, 2.3 mmol), Intermediate 6-d (1.1 g, 3.5 mmol) and potassium carbonate (964 mg, 7.0 mmol) in DME (12.4 ml) and water (3.1 ml) was added PdCl₂(dppf) (170 mg, 0.2 mmol) and the reaction was heated in a pressure vessel at 105° C. overnight and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided Intermediate 10-a as a beige solid.

### Step 2: Intermediate 10-b

To a solution of Intermediate 10-a (1.2 g, 2.4 mmol) in 1,4-dioxane (10 ml) and methanol (1 ml) cooled to 0° C. was added a solution of 4N HCI in 1,4-dioxane (11.9 ml, 47.7 mmol). After the addition was completed the reaction was stirred for 1 hour at 0° C. Diethyl ether was added, a precipitate formed and was collected by filtration to provide Intermediate 10-b·2HCl as a white solid.

### Synthesis of Compound 1:

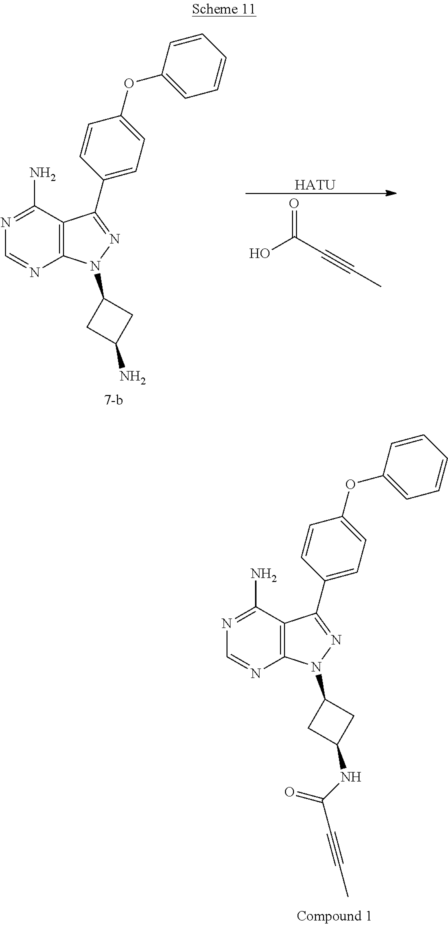

To a solution of Intermediate 7-b·2HCl (547 mg, 1.2 mmol) in DMF (8 ml) were sequentially added HATU (560 mg, 1.5 mmol) and but-2-ynoic acid (103 mg, 1.2 mmol), and the reaction was then stirred at room temperature for 1 hour. A saturated aqueous solution of ammonium chloride and ethyl acetate were added, the organic layer was separated, washed with brine, dried over MgSO₄, filtered and concentrated under reduced pressure. Purification by silica gel chromatography provided Compound 1 as a white solid.

Compounds 4 and 7 were obtained in a similar manner to Compound 1 starting from Intermediate 8-b·2HCl and 10-b·2HCl respectively.

### Synthesis of Compound 2:

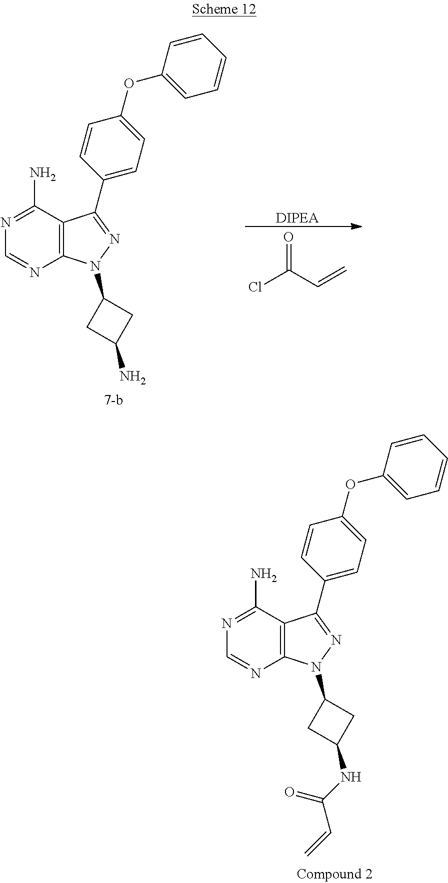

To a solution of Intermediate 7-b·2HCl (2.0 g, 5.4 mmol) in dichloromethane (20.0 ml) cooled to −78° C. were sequentially added TEA (7.5 ml, 53.7 mmol) and acryloyl chloride (434 μl, 5.4 mmol), and the reaction was then stirred at −78° C. for 3 hours. A saturated aqueous solution of ammonium chloride and ethyl acetate were added, the organic layer was separated, washed with brine, dried over MgSO₄, filtered and concentrated under reduced pressure. Purification by silica gel chromatography provided Compound 2 as a white solid.

Compounds 3, 5 and 6 were obtained in a similar manner to Compound 2 starting from Intermediate 9-b·2HCl, 8-b·2HCl and 10-b·2HCl respectively.

TABLE 1
Example Compounds of Formula I

| Com- | | |
|---|---|---|
| pound | Structure | MS (m/z) |
| 1 | 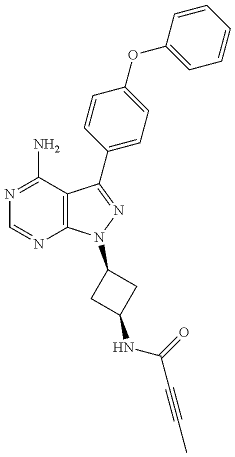 [SMILES: CC#CC(=O)N[C@H]1C[C@@H](n2nc(-c3ccc(Oc4ccccc4)cc3)c3c(N)ncnc32)C1] | [M + H]⁺ = 439.2 |
| 2 | 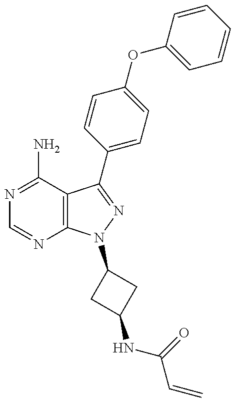 [SMILES: C=CC(=O)N[C@H]1C[C@@H](n2nc(-c3ccc(Oc4ccccc4)cc3)c3c(N)ncnc32)C1] | [M + H]⁺ = 427.2 |
| 3 | 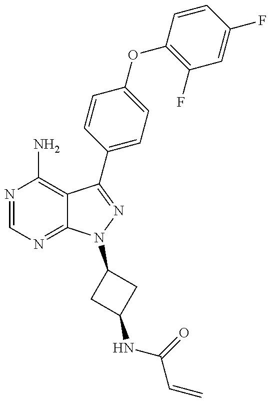 [SMILES: C=CC(=O)N[C@H]1C[C@@H](n2nc(-c3ccc(Oc4ccc(F)cc4F)cc3)c3c(N)ncnc32)C1] | [M + H]⁺ = 463.2 |
| 4 | 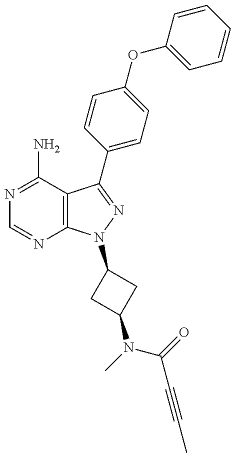 [SMILES: CC#CC(=O)N(C)[C@H]1C[C@@H](n2nc(-c3ccc(Oc4ccccc4)cc3)c3c(N)ncnc32)C1] | [M + H]⁺ = 453.3 |
| 5 | 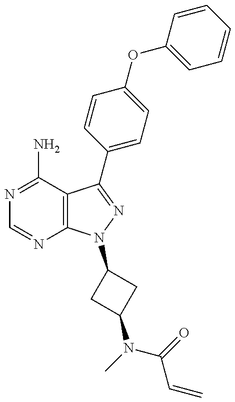 [SMILES: C=CC(=O)N(C)[C@H]1C[C@@H](n2nc(-c3ccc(Oc4ccccc4)cc3)c3c(N)ncnc32)C1] | [M + H]⁺ = 441.3 |
| 6 | 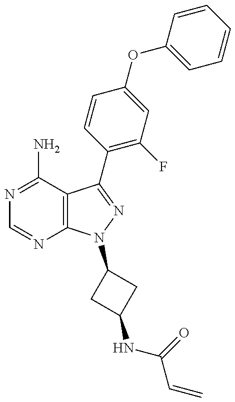 [SMILES: C=CC(=O)N[C@H]1C[C@@H](n2nc(-c3ccc(Oc4ccccc4)cc3F)c3c(N)ncnc32)C1] | [M + H]⁺ = 445.2 |
| 7 | 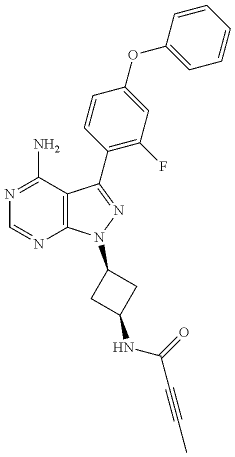 [SMILES: CC#CC(=O)N[C@H]1C[C@@H](n2nc(-c3ccc(Oc4ccccc4)cc3F)c3c(N)ncnc32)C1] | [M + H]⁺ = 457.2 |

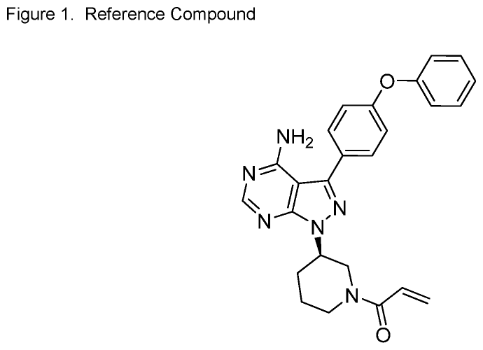

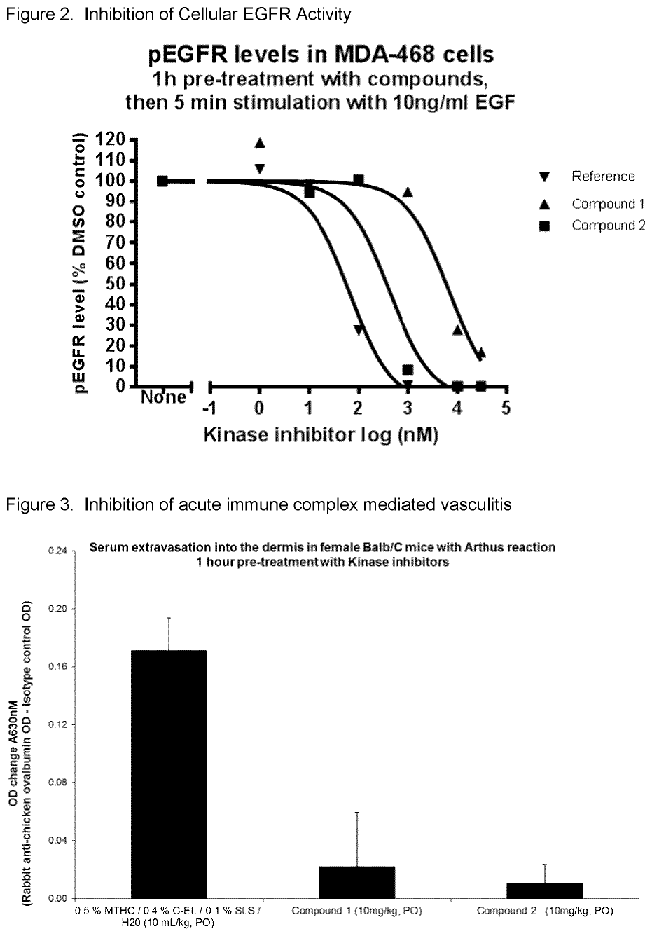

Assays for determining kinase activity are described in more details in the accompanying examples. The Reference compound used is shown in FIG. 1, it is disclosed in PCT publication WO 2008/039218 A2 and identified as 1-((R)-3-(4-amino-3-(4-phenoxyphenyl)-1H-pyrazolo[3,4-d]pyrimidin-1-yl)piperidin-1-yl)prop-2-en-1-one.

### EXAMPLE 1

### Kinase Inhibition

### BTK, EGFR and Erb2 Kinase Inhibition Assays

In vitro potency of selected compound was defined against human BTK, EGFR, and ErbB2 kinases using Kinase Profiler radiometric protein kinase assays performed at Eurofins Pharma Discovery Services UK Limited.

Each kinase is diluted in buffer and all compounds were prepared to 50x final assay concentration in 100% DMSO. This working stock of the compound was added to the assay well as the first component in the reaction, followed by the remaining components as detailed in the assay protocol listed above. The reaction was initiated by the addition of the MgATP mix. The kinase reaction was performed at room temperature for 40 minutes in presence of 250 μM substrate, 10 mM Mg Acetate, [γ-33P-ATP] (specific activity approx. 500 cpm/pmol, concentration as required) and variable test article concentrations. The ATP concentrations in the assays were within 15 μM of the apparent. The reaction was stopped by the addition of 3% phosphoric acid solution. 10 μL of the reaction is then spotted onto a P30 filtermat and washed three times for 5 minutes in 75 mM phosphoric acid and once in methanol prior to drying and scintillation counting. In addition positive control wells contain all components of the reaction, except the compound of interest; however, DMSO (at a final concentration of 2%) were included in these wells to control for solvent effects as well as blank wells contain all components of the reaction, with a reference inhibitor replacing the compound of interest. This abolishes kinase activity and establishes the base-line (0% kinase activity remaining). The potency of each compound was reported by estimating the EC₅₀.

Data in Table 2 and Table 3 indicate that compounds of the instant invention are potent BTK inhibitors but poorly inhibit EGFR and ErbB2. In contrast, the reference compound (FIG. 1) inhibits BTK but is also a potent an inhibitor of EGFR and ErbB2. These findings suggest that compounds of the present invention will be effective in treating BTK-associated disorders and exhibit reduced non-specific adverse effects due to EGFR and/or ErbB2 inhibition.

TABLE 2
Results of BTK Kinase Inhibition

| | Kinase Inhibition |
|---|---|
| | IC₅₀ (nM) |
| Compound | BTK |
| Reference | 4 |
| 1 | 3 |
| 2 | 3 |
| 3 | 10 |
| 4 | 1 |
| 5 | 0.7 |
| 6 | 3 |
| 7 | 1 |

TABLE 3
Results of EGFR
and ErB2 inhibition

| | Kinase | |
|---|---|---|
| | Inhibition | |
| | IC₅₀ (nM) | |
| Compound | EGFR | ErbB2 |
| Reference | 17 | 30 |
| 1 | >1000 | >1000 |
| 2 | >1000 | 97 |

### EXAMPLE 2

### Splenic Cell Proliferation Assay

Proliferation of splenocytes in response to anti-IgM can be blocked by inhibition of BTK. Splenocytes were obtained from 6 week old male CD1 mice (Charles River Laboratories Inc.). Mouse spleens were manually disrupted in PBS and filtered using a 70um cell strainer followed by ammonium chloride red blood cell lysis. Cells were washed, resuspended in Splenocyte Medium (HyClone RPMI supplemented with 10% heat-inactivated FBS, 0.5X non-essential amino acids, 10 mM HEPES, 50 μM beta mercaptoethanol) and incubated at 37° C., 5% CO₂ for 2h to remove adherent cells. Suspension cells were seeded in 96 well plates at 50,000 cells per well and incubated at 37° C., 5% CO₂ for 1h. Splenocytes were pre-treated in triplicate with 10,000 nM curves of Formula 1 compounds for 1h, followed by stimulation of cell proliferation with 2.5 μg/ml anti-IgM

F(ab′)₂ (Jackson ImmunoResearch) for 72 h. Cell proliferation was measured by Cell Titer-Glo Luminescent Assay (Promega). EC₅₀ values (50% proliferation in the presence of compound as compared to vehicle treated controls) were calculated from dose response compound curves using GraphPad Prism Software. EC₅₀ values are reported in Table 3. Data presented in Table 4 demonstrates that compounds of the instant invention are potent inhibitors of B-cell receptor mediated proliferation which is dependent on BTK and suggest that inventive compounds can be effective in the treatment of diseases characterized by B-cell dysfunction including autoimmune disease and inflammation.

TABLE 4
Results of inhibition of
splenic cell proliferation

| | Spleen cell |
|---|---|
| Compound | EC₅₀ (nM) |
| Reference | 0.4 |
| 1 | 0.2 |
| 2 | 0.3 |
| 3 | 0.9 |
| 4 | 0.8 |
| 5 | 0.5 |
| 6 | 0.4 |
| 7 | 0.4 |

### EXAMPLE 3

### TMD-8 Survival Assay

TMD-8 human activated B cell diffuse large B cell lymphoma cells were seeded in 96-well plates at a density of 20,000 cells/well in HyClone RPMI supplemented with 10% FBS (Fisher)/1% Penicillin/Streptomycin (HyClone) and incubated at 37° C., 5% CO₂. Cells were treated in triplicate with 1,000 nM or 100 nM curves of compounds for 72h. Cell survival was measured by Cell Titer-Glo Luminescent Assay (Promega). EC₅₀ values (50% proliferation in the presence of compound as compared to vehicle treated controls) were calculated from dose response compound curves using GraphPad Prism Software. Data presented in Table 5 demonstrates that compounds of the instant invention potently affect the survival of TMD-8 tumor cells and suggest that inventive compounds can be effective in treatment of cancer.

TABLE 5
Results of TMD-8
survival assay

| | TMD8 |
|---|---|
| Compound | EC₅₀ (nM) |
| Reference | 2.0 |
| 1 | 1.0 |
| 2 | 1.9 |
| 3 | 6.5 |
| 4 | 5.4 |
| 5 | 1.9 |
| 6 | 3.0 |
| 7 | 1.5 |

### EXAMPLE 4

### Inhibition of cellular EGFR

EGFR autophosphorylation was measured in MDA-468 human breast tumor cells which express functional EGFR. Cells were plated at a densitity of 400 000 cells/well in 6×12-well plates in a final volume of 1 ml/well in DMEM high glucose+10% FBS +1% Pen/Strep and cultured at 37 C and 5% CO₂ overnight. The following day the media was replaced with serum free media and the cells were cultured for a further 24 hours.

Cells were pretreated with compound for 1 hour and then stimulated with 10 ng/mL EGF for 5 minutes. Media was removed and cells were lysed in 100 uL/well of RIPA buffer+1% Protease Inhibitor cocktail+1% Phosphatase inhibitor cocktail. Protein concentrations were determined by

BCA assay. 25μ of cellular protein from each cell treatment was separated by SDS-PAGE. Proteins were transferred to nitrocellulose membranes and blocked with TBS-T+5% non-fat milk. EGFR and phospho-EGFR were detected following incubation overnight at 4° C. with Rabbit anti-phospho-EGFR (Tyr1068) (Cell signaling) diluted 1/1000 and Mouse anti-EGFR (1F4) (Cell signaling) diluted 1/1000 in TBS-T+5% nonfat dry milk and then incubation for 1 h at RT with IR Dye 800CW goat anti-rabbit diluted 1/15000 and with IR Dye 680RD goat anti-mouse diluted 1/15000 in TBS-T+5% nonfat dry milk+0.01% SDS. Antibody binding was quatified using a Li-cor Odyssey CLx. Phospho-EGFR/EGFR ratios were calculated for each test condition of control and test article and the EC50 of inhibiton of EGF-stimulated EGFR phosphorylation was calculated.

Data presented in Table 6 demonstrates that the reference compound (see FIG. 1) potently inhibits cellular EGFR function and that compounds of the instant invention have much less effect on EGFR in cells. These data suggest that compounds of the instant invention can result in fewer EGFR-related adverse effects.

TABLE 6
Inhibition of EGFR in cells

| | EGFR |
|---|---|
| | Inhibition |
| | EC₅₀ |
| Compound | (nM) |
| Reference | 56 |
| 1 | 5956 |
| 2 | 376 |

### EXAMPLE 5

### Formation of Glutathione Adducts

Generation of non-specific thiol adducts can be measured by assaying reactivity of compounds with GSH. Formation of GSH adducts was measured by incubating GSH (10 mM) in 100 mM Phosphate Buffer pH 7.4 with test article at 20 micromolar final concentration. Briefly, 600 microliters of phosphate buffer pH 7.4 was added in a glass vial with 200 microliters of 100 micromolar test article in DMSO. Samples were warmed at 37° C. for 10 minutes and the reaction was initiated by the addition of 200 microliters of 50 mM reduced GSH in phosphate buffer pH 7.4 and incubated at 37° C. At various time points (0.5 min, 5 min, 15 min, 30 min, 60 min, 120 min and 180 min) 50 microliter aliquots were placed into an injection vial equipped with an insert and mixed with 50 microliters of 1% formic acid in 99% Methanol. Vials were loaded onto the HPLC injection tray and each sample was injected onto a C18 column (ACE 3 C18, 4.6×50 mm column) and both the parent and product were monitored by UV absorbance at 254 nm on an Agilent Technologies 1100 series HPLC. Peaks were integrated and the percentage of product present calculated as the percent of the total peak area on the parent and product at 60 minutes.

Data show that compounds of the instant invention form fewer non-specific glutathione adducts than the reference compound (Table 7).

TABLE 7
Formation of glutathione adducts

| | GSH Reactivity |
|---|---|
| | GSH Conjugate |
| | at 60 minutes |
| Compound | (% of total Peaks) |
| Reference | 31.7 |
| 1 | 7.3 |
| 2 | 14.2 |
| 3 | 12.6 |
| 4 | 8.4 |
| 5 | 28.9 |
| 6 | 12.6 |
| 7 | 5.9 |

### EXAMPLE 6

### Human Liver Microsome Stability

Intrinsic liver microsomal stability was determined using cryopreserved human liver microsomes. Test articles were incubated at 37° C. at a final concentration of 1 μM with 0.5 mg microsomes in the presence of 0.5mM NADPH in at total volume of 480 μL PBS. At various time points 50 μL aliquots were removed and mixed with 100 μL of methanol. Following centrifugation 100 μL of the supernatant was transferred to a 96-well plate and analyzed by LC-MS/MS using test-article specific methods. Controls included absence of microsomes or co-factors or use of heat-denatured microsomes. Intrinsic clearance was calculated using the formula: (In(% Time 0/% Time 1)/T1−T0)×Volume of incubation/protein in the incubation. Table 8 shows that compounds of the instant invention are more stable in human liver microsomes than the reference compound.

TABLE 8
Stability in human
liver microsomes

| | Human |
|---|---|
| | Liver |
| | Microsomes |
| | Intrinsic |
| | clearance |
| Compound | (μL/min/mg) |
| Reference | 253 |
| 1 | 34.9 |
| 2 | 15.6 |

### Mouse Pharmacokinetics

Plasma pharmacokinetic studies were conducted in CD1 mice to compare the plasma exposure following oral and intravenous administration of the free-base form of each compound to mice and to calculate bioavailability. Male CD-1 mice (25-30 g on delivery; Charles River) were used for mouse PK studies. The mice were housed 3 per cage in a room under controlled conditions of temperature (20-250° C.) and humidity (40-70%), with a 12 h dark/light cycle. The animals were acclimated for a minimum of 3 days prior to use and received standard rodent chow (Charles River) and municipal tap water ad libitum. Nine animals were used per study, they were dosed PO or IV and bled from the mandibular vein, 2 to 3 times per animal at the following time points; pre dose, 15 and 30 min, 1, 2, 3, 5, 7 and 24 h (0.1 mL whole blood /time point; composite PK). Blood was collected into microvette K3E tubes (SARSTEDT) at RT and then centrifuged at 5,000 RPM for 2 min at 4° C. in order to separate plasma. Isolated plasma (0.015 mL/time point) was pipetted into 96 well plates (Canadian Life Science) which were stored at minus 20° C. until analysis (2 duplicate plates per study).

Concentrations of compound in rodent plasma samples were determined by LC-MS-MS. Two standard curves containing 8 points each were prepared by spiking in methanol or plasma with a solution containing the test article at a defined concentration. The range of final compound concentration in the standard curve was 0 to 1 μg/mL. Three sets of quality control (QC) samples in plasma at low, medium and high concentration within the standard curve range were also prepared. For analysis, the compound was extracted using protein precipitation extraction procedure. Briefly, a three-fold volume of methanol containing an internal standard was added to the plasma and standard curve solution samples and then incubated at 4° C. for 5 minutes. The plates were centrifuged for 30 minutes at 4° C. at 4000 rpm and the resulting supernatant was transferred to a 96-well propylene plates and sealed with cover to avoid sample evaporation. Ten μL aliquots of extracted samples were injected using a 96-well plate autosampler held at 4° C. onto a ACE C18 50×4.6 mm, 3 um HPLC column held at 30° C. Plasma derived parent compound was eluted using the following conditions: pump flow rate was set at 1 mL/min for a five minutes chromatographic method using a 3 min gradient elution from 20% solvent A (0.1% formic acid in water) and 80% solvent B (0.1% Formic Acid in Methanol) to 10% solvent A and 90% solvent B. The method was followed by 1 min column wash at 95% solvent B and 1 min column re-equilibration back to 80% solvent B. Under these conditions the compound was eluted after 1.8 minutes. For LC-MS-MS compound detection, the curtain gas was set at 10, collision gas was set at 8, ion spray voltage was set at 4500, temperature was set at 500, ion source gas 1 was set at 40 and ion source gas 2 was set at 60. Using a positive ionization mode, the MRM transition monitored was set at 567.25→1388.600. Under these conditions, standard curves were linear up to 2000 ng/mL, the lower limit of quantification was generally at 10 ng/mL.

The plasma concentration of each compound was calculated for each sample by integration of the peak area with reference to the standard curve. The area under the curve (AUCtot and AUClast), the maximum plasma concentration (Cmax) and time (Tmax), and terminal half life (T1/2), clearance and volume of distribution were calculated by regression analysis using Kinetica version 5.0 (Thermo Fisher Scientific) using a Non-Compartmental Extravascular/IV Bolus analysis model. Data shows that compounds of the instant invention have greater bioavailability than the reference compound in mice (Table 9).

TABLE 9
Pharmacokinetics in mice

| | Plasma Pharmacokinetics | | | |
|---|---|---|---|---|
| | Intravenous | Oral | | |
| | 3 mg/kg | 10 mg/kg | | Bio- |
| | AUC | Cmax | AUC | availability |
| Compound | (ng*hr/mL) | (ng/mL) | (ng*hr/mL) | % |
| Reference | 1070 | 219 | 349 | 9.8 |
| 1 | 841 | 1283 | 1454 | 52 |
| 2 | 954 | 1351 | 1435 | 45 |

### Mouse Arthus

Formation and reaction to immune complexes is characteristic of antibody mediated autoimmune and inflammatory disease. BTK is important in signaling pathways downstream of Fc receptor stimulation which can be modeled by immune complex mediated acute vasculitis. Mouse studies were conducted as reported in Braselmann S, et al. J Pharmacol Exp Ther, 2006, 319:998-1008.

In summary, female Balb/c mice (6-7 weeks on arrival) were habituated to the animal facility for at least 4 days. On the day of the experiment, animals were pre-treated (t=minus 1 h) with compound or vehicle alone by gavage (PO). At t=0, animals were injected intravenously (IV; 0.1 mL/mouse) with saline containing chicken ovalbumin and Evan's blue (10 mg/mL of each). Ten minutes later (t=10 min), animals were anesthesized with isoflurane, the dorsal surface was shaved and rabbit anti-chicken ovalbumin antibody was then injected intradermally at one site on the right side of the animal (25 μg in 30 μL). The same amount of isotype control antibody was then injected on the left side.

The animals were then returned to their home cage and skin punches (8 mm) were collected from each injection site four hours later. The samples were placed in 1 mL formamide overnight at 80° C. (1 skin biopsy per 1 mL formamide in a glass tube). The amount of Evan's blue in the formamide solution was then assessed by spectrophotometry (630 nm) as a measure of serum extravasation into the dermis.

Compounds 1 and 2 demonstrated efficacy when administered by oral gavage at 10 mg/kg and suppressed immune complex mediated vasculitis by 87% and 94% respectively (see FIG. 3).

### Mouse CIA

Mouse CIA model was performed using the methods described by Trentham D E, Townes A S, Kang A H. Autoimmunity to Type II Collagen: An Experimental Model of Arthritis. J Exp Med 1977; 857-868, and Bendele A M. Animal Models of Rheumatoid Arthritis. J Musculoskel Interact 2001; 377-385.

In summary, male B10R111 mice (7-9 wks on arrival) were habituated to the animal facility for at least 4 days. On experimental day 0 mice were anaesthetized with isoflurane and the dorsal surface was shaved. Collagen, emulsified in Freund's complete adjuvant (CFA) supplemented with additional mycobacterium tuberculosis (TB) H37Ra, was injected intradermally at the base of the tail (0.15 mL / animal; 2 mg/mL collagen and 2.5 mg/mL TB in CFA). This CFA treatment was repeated on day 15.

From day 15 to the end of the study animals were scored daily for signs of arthritis. On the first day of disease (RA Day 1) animals were recruited to the study and grouped using a balanced design based on arthritis score. Once recruited, animals were weighed and dosed twice daily by gavage (PO, BID). Recruited animals were then scored twice a week on RA days 1, 5, 8 and 12.

At the end of the study (RA day 12) animals were weighed and scored.

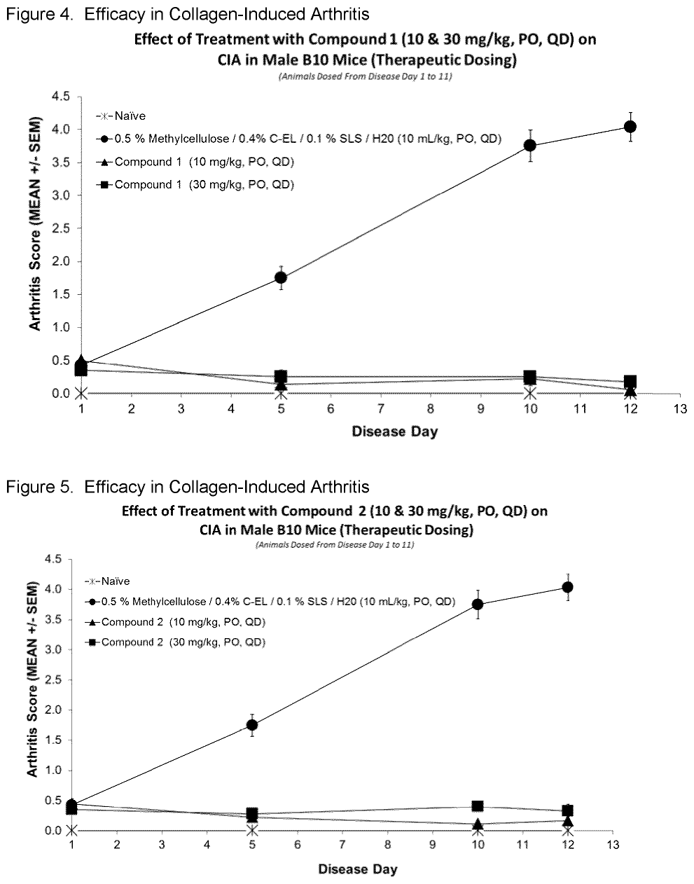

Compounds 1 and 2 prevented the progression of arthritis when administered by oral gavages at 10 and 30 mg/kg (see FIG. 4 and FIG. 5).

### Antitumor Activity Study

Female C.B-17/IcrHsd-PrkdcsidLystbg-J mice (Scid mice; Harlan, 6-8 wk on delivery) were used for these studies. The mice were housed 4 per cage in a ventilated rack in a room under controlled conditions of temperature (20-25° C.) and humidity (40-70%), with a 12 h dark/light cycle. The animals were fed ad libitum with irradiated rodent diet (Harlan) and received autoclaved tap water. The cage, bedding and enrichment materials inside the home cage were autoclaved prior to use and all cage and animal manipulations were carried out inside a sterile laminar flow hood. Animals were acclimated for 1 week prior to cell injection.

TMD-8 human activated B cell diffuse large B cell lymphoma cells were grown in HyClone RPMI supplemented with 10% FBS (Fisher)/1% Penicillin/Streptomycin (HyClone) at 37° C., 5% CO₂ and then prepared for injection. On day 0 (d0), cells were suspended at 2×10⁸ cells/mL in PBS containing 10% FBS. The cell suspension was combined 1:1 with Matrigel (VWR) and 0.1 mL of cell suspension (1×10⁷ cells) was injected (25 gauge needle) subcutaneously into the shaved right flank of mice under isoflurane anesthesia. All manipulations were carried out inside a laminar flow hood.

When the mean tumor volume reached 200-300 mm³ (d21) mice were randomized into groups of 10 based on tumor size and treatment was initiated. Animals were then dosed once daily by oral gavage (10 mL/kg). General condition and BW of all mice was assessed daily and tumor measurements collected twice per week (Mon and Thur). Any animals with tumors of 2000 mm³ and above were euthanized.

The long (a) and short (b) axis of tumors were measured with electronic calipers (Mitutoyo) and tumor volume (mm³) was calculated (a*b²/2) and temporal changes in tumor volume and body weight were assessed day 21 (d21) to day 38 (d28).

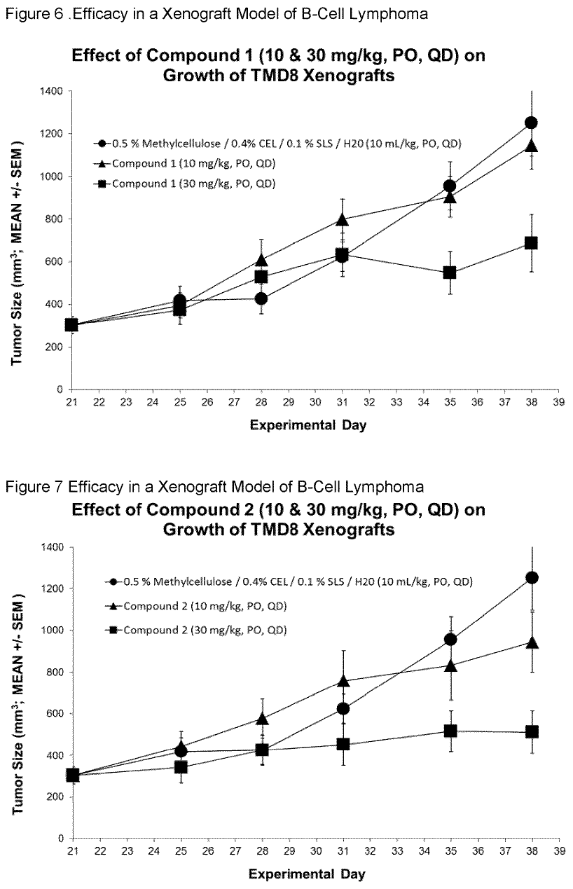

Compound 1 (see FIG. 6) and compound 2 (see FIG. 7) reduce growth of TMD-8 xenograft B-cell lymphoma in mice.

1. A compound of Formula I: 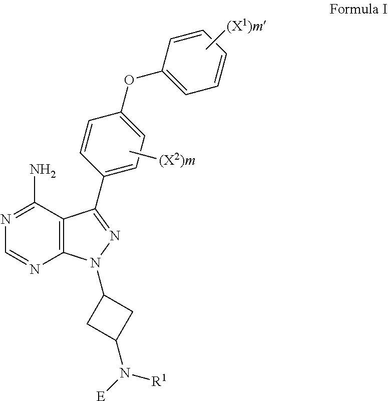
or pharmaceutically acceptable salt, stereoisomer, tautomer, isotope, prodrug, complex or biologically active metabolite thereof, wherein
X¹ and X² are independently selected from the group consisting of hydrogen and halogen;
m is an integer from 0 to 4;
m′ is an integer from 0 to 5;
R¹ is selected from hydrogen, or a substituted or unsubstituted alkyl; and
E is: 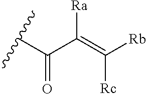
wherein Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl , or
Ra and Rb optionally can be fused with the carbon atoms to which they are attached to form a 3- to 8-membered substituted or unsubstituted cycloalkyl ring, or a 3- to 8-membered substituted or unsubstituted heterocyclyl ring, or
Rb and Rc optionally can be fused with their intervening carbon atom to form a 3- to 8-membered substituted or unsubstituted cycloalkyl ring, or a 3- to 8-membered substituted or unsubstituted heterocyclyl ring, or
Ra and Rb optionally form a triple bond.

2. [[A]]The compound of Formula I having the stereochemical configuration of Formula II: 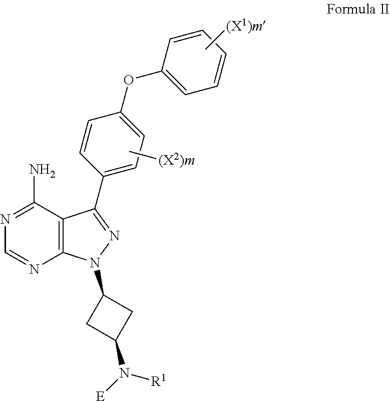
or pharmaceutically acceptable salt, stereoisomer, tautomer, isotope, prodrug, complex or biologically active metabolite thereof.

3. The compound according to claim 1 or claim 2, wherein the halogen is fluorine.

4. The compound according to claim 1, wherein R¹ is hydrogen or methyl.

5. The compound according to claim 1, wherein E is:

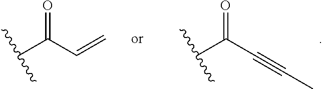

6. The compound according to claim 1, wherein m and m′ is an integer from 0 to 2.

7. The compound of claim 1 selected from the group consisting of:

| Compound | Structure |
|---|---|
| 1 | 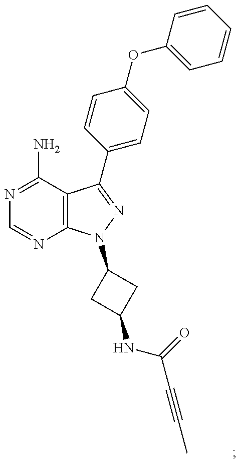 [SMILES: CC#CC(=O)N[C@H]1C[C@@H](n2nc(-c3ccc(Oc4ccccc4)cc3)c3c(N)ncnc32)C1] |
| 2 | 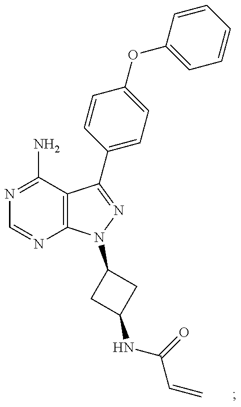 [SMILES: C=CC(=O)N[C@H]1C[C@@H](n2nc(-c3ccc(Oc4ccccc4)cc3)c3c(N)ncnc32)C1] |
| 3 | 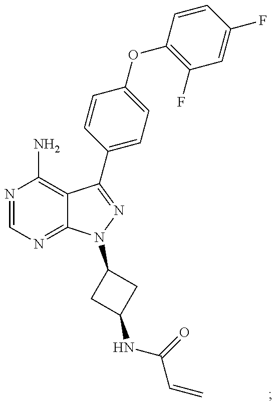 [SMILES: C=CC(=O)N[C@H]1C[C@@H](n2nc(-c3ccc(Oc4ccc(F)cc4F)cc3)c3c(N)ncnc32)C1] |
| 4 | 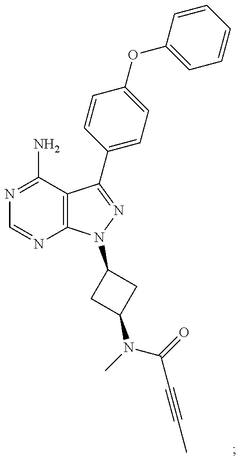 [SMILES: CC#CC(=O)N(C)[C@H]1C[C@@H](n2nc(-c3ccc(Oc4ccccc4)cc3)c3c(N)ncnc32)C1] |
| 5 | 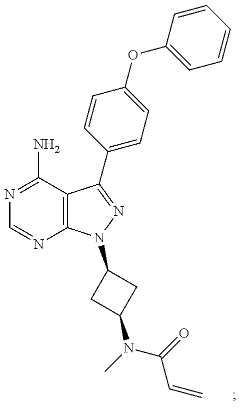 [SMILES: C=CC(=O)N(C)[C@H]1C[C@@H](n2nc(-c3ccc(Oc4ccccc4)cc3)c3c(N)ncnc32)C1] |
| 6 | 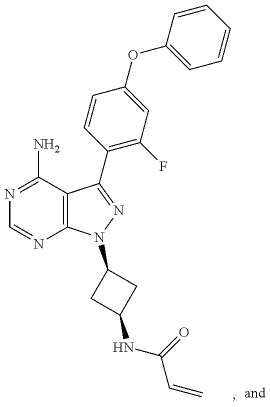 [SMILES: C=CC(=O)N[C@H]1C[C@@H](n2nc(-c3ccc(Oc4ccccc4)cc3F)c3c(N)ncnc32)C1] |
| 7 | 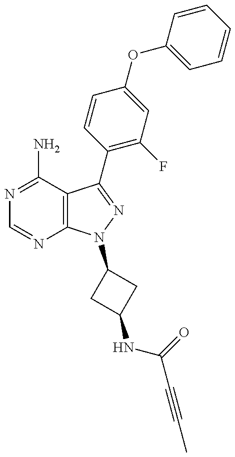 [SMILES: CC#CC(=O)N[C@H]1C[C@@H](n2nc(-c3ccc(Oc4ccccc4)cc3F)c3c(N)ncnc32)C1] |

or a pharmaceutically acceptable salt, stereoisomer, tautomer, isotope, prodrug, complex or biologically active metabolite thereof.

8-20. (canceled)

21. A pharmaceutical composition comprising the compound or a pharmaceutically acceptable salt, stereoisomer, tautomer, isotope, prodrug, complex or biologically active metabolite thereof of claim 1 in combination with one or more pharmaceutically acceptable excipient, diluent or carrier.

22-33. (cancelled)

34. A method for treating a subject suffering from a protein kinase mediated disease or condition, comprising administering to the subject a therapeutically effective amount of a compound of Formula I, 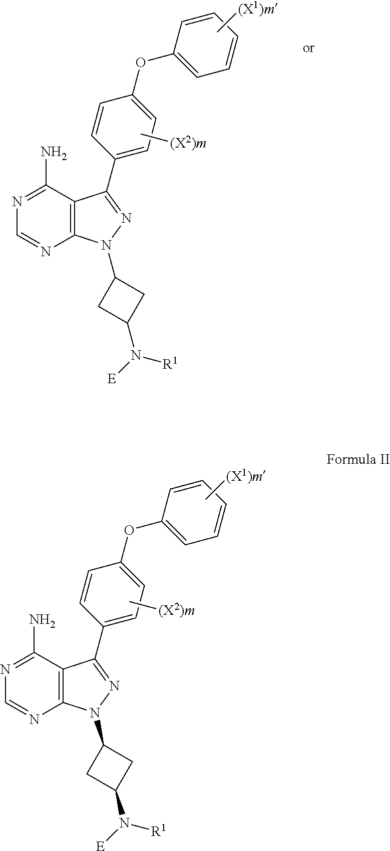
or a pharmaceutically acceptable salt, stereoisomer, tautomer, isotope, prodrug, complex or biologically active metabolite thereof, wherein
X¹ and X² are independently selected from hydrogen or halogen;
m is an integer from 0 to 4;
m′ is an integer from 0 to 5;
R¹ is selected from hydrogen or a substituted or unsubstituted alkyl; and
E is: 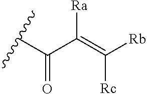
wherein Ra, Rb and Rc are independently selected from hydrogen, halogen, -CN, substituted or unsubstituted alkyl, substituted or unsubstituted heteroalkyl, substituted or unsubstituted cycloalkyl, or substituted or unsubstituted heterocyclyl , or
Ra and Rb optionally can be fused with the carbon atoms to which they are attached to form a 3- to 8-membered substituted or unsubstituted cycloalkyl ring, or a 3- to 8-membered substituted or unsubstituted heterocyclyl ring, or
Rb and Rc optionally can be fused with their intervening carbon atom to form a 3- to 8-membered substituted or unsubstituted cycloalkyl ring, or a 3- to 8-membered substituted or unsubstituted heterocyclyl ring, or
Ra and Rb optionally form a triple bond.

35. The method of claim 34, wherein the disease, disorder or condition is associated with a TEC kinase family member.

36. The method of claim 34, wherein the disease, disorder or condition is associated with BTK kinase activity.

37. The method according to claim 34, wherein the disease or condition is cancer, autoimmune disease, allergic disease, inflammatory disease, graft-versus-host disease, thromboembolic disease, neurological disorder, infectious disease, viral infection, bone-related disease or a combination thereof.

38. A method of reducing the enzymatic activity of BTK in a subject suffering from cancer, autoimmune disease, allergic disease, inflammatory disease, viral infection or a combination thereof, comprising administering to the subject a therapeutically effective amount of the compound of claim 1, or a pharmaceutically acceptable salt, stereoisomer, tautomer, isotope, prodrug, complex or biologically active metabolite thereof.

39. The method according to claim 37, wherein the disease or condition is:
(a) selected from the group consisting of rheumatoid arthritis, psoriatic arthritis, lupus, uveitis, myasthenia gravis, warm autoimmune hemolytic anemia, Wegener's granulomatosis, Sjogren's disease, Sjogren's dry eye, non-Sjogren's dry eye disease, psoriasis, and asthma; or
(b) a B-cell proliferative disorder; or
(c) a proliferative disorder selected from the group consisting of diffuse large B cell lymphoma, follicular lymphoma, chronic lymphocytic lymphoma, chronic lymphocytic leukemia, B-cell prolymphocytic leukemia, small lymphocytic lymphoma (SLL), multiple myeloma, non-Hodgkin lymphoma, Hodgkin-lymphoma, myelofibrosis, lymphoplasmacytic lymphoma, Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, B-ALL, and lymphomatoid granulomatosis; or
(d) inflammatory bowel disease, arthritis, rheumatoid arthritis, psoriatic arthritis, osteoarthritis, Still's disease, juvenile arthritis, type I diabetes, myasthenia gravis, Hashimoto's thyroiditis, Ord's thyroiditis, Basedow's disease, Sjogren's syndrome, multiple sclerosis, Guillain- Barre syndrome, acute disseminated encephalomyelitis, Addison disease, opsoclonus-myoclonus syndrome, ankylosing spondylitis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's disease, Takayasu arteritis, temporal arteritis, warm autoimmune hemolytic anemia, Wegener granuloma, psoriasis, alopecia universalis, Burchett disease, chronic fatigue syndrome, dysautonomia, endometriosis, interstitial cystitis, myotonia, vulvodynia, pemphigus , systemic lupus erythematosus, asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, epicondylitis, epididymitis, fasciitis, fibrositis, gastritis, gastroenteritis, hepatitis, hidradenitis suppurativa, laryngitis, mastitis, meningitis, myelitis, myocarditis, myositis nephritis, oophoritis, orchitis, osteitis, pancreatitis, parotitis, pericarditis, peritonitis, pharyngitis, pleuritis, phlebitis, pneumonia, proctitis, prostatitis, pyelonephritis, rhinitis, salpingitis, sinusitis, stomatitis, synovitis, tendinitis, tonsillitis, uveitis, vaginitis, vasculitis vulvitis, non-Hodgkin's lymphomas, Burkitt's, lymphoma, AIDS-related lymphoma, marginal zone B-cell lymphoma, nodal marginal zone B cell lymphoma,extranodal marginal zone B-cell lymphoma, splenic marginal zone B-cell lymphoma, diffuse large B-cell lymphoma, primary effusion lymphoma, lymphoma-like granulomatous disease, follicular lymphoma, B-cell chronic lymphocytic leukemia, B cell prolymphocytic leukemia, lymphoplasmacytic leukemia/Waldenstrom's macroglobulinemia, plasmacytoma, mantle cell lymphoma, mediastinal large B-cell lymphoma, intravascular large B-cell lymphoma, hairy cell leukemia, pancreatic endocrine tumors and multiple myeloma; or
(e) a viral infection that is HIV/AIDS.

40-43. (canceled)

44. A method of modulating kinase activity function and/or inhibiting protein kinase activity in a subject comprising administering a therapeutically effective amount of the compound of claim 1 or a pharmaceutically acceptable salt, stereoisomer, tautomer, isotope, prodrug, complex or biologically active metabolite thereof, to said subject to modulate and/or inhibit the enzymatic activity of a protein kinase.

45. A method of inhibiting protein kinase in a cell or tissue comprising contacting the cell or tissue with an effective amount of the compound, or a pharmaceutically acceptable salt, stereoisomer, tautomer, isotope, prodrug, complex or biologically active metabolite thereof, according to claim 1.

46. (canceled)

47. The method according to claim 44, wherein said kinase activity function is associated with TEC kinase family members activity, wherein said TEC kinase family member optionally is BTK.

48. (canceled)

49. A method of reducing the enzymatic activity of BTK comprising contacting the BTK enzyme with an effective amount of the compound of claim 1.

50. The method according to claim 34, further comprising the administration of a therapeutically effective amount of at least one additional active pharmaceutical ingredient for the treatment of cancer, autoimmune diseases, allergic diseases, inflammatory diseases or viral infection in combination therapy, wherein the additional active pharmaceutical ingredient is optionally selected from the group comprising steroids, leukotriene antagonists, anti-histamines, anti-cancer, anti-viral, anti-biotic agents, protein kinase inhibitors, immune modulators, checkpoint inhibitors and a combination thereof.

51. (canceled)

52. A probe comprising the compound of claim 1 or a pharmaceutically acceptable salt, stereoisomer, tautomer, isotope, prodrug, complex or biologically active metabolite thereof and a detectable label or affinity tag for said compound, wherein the detectable label is optionally selected from the group consisting of: a fluorescent moiety, a chemiluminescent moiety, a paramagnetic contrast agent, a metal chelate, a radioactive isotope-containing moiety and biotin.

53-63. (canceled)
